(19) United States
(12) Patent Application Publication
(10) Pub. No.: US 2008/0166329 A1
(43) Pub. Date: Jul. 10, 2008

(54) Medical device and methods for living cell injection

(76) Inventors: Hsing-Wen Sung, Hsinchu (TW); Hosheng Tu, Newport Beach, CA (US)

Correspondence Address: HOSHENG TU, 15 RIEZ, NEWPORT BEACH, CA, 92657-0116, US

(21) Appl. No.: 11/986,708

(22) Filed: Nov. 26, 2007

Related U.S. Application Data
(63) Continuation-in-part of application No. 11/256,729, filed on Oct. 24, 2005.
(60) Provisional application No. 60/861,157, filed on Nov. 27, 2006.
(60) Provisional application No. 60/906,690, filed on Mar. 13, 2007.

(51) Int. Cl. A61K 35/12; A61M 31/00; A61M 25/00; A61P 43/00

(52) U.S. Cl. 424/93.7; 604/506

(57) ABSTRACT

A novel method, using a thermoreversible MC/PBS/Collagen hydrogel coated on the TCPS dish, for harvesting a living cell sheet or spheroid with ECM. In one application, the obtained living cell sheet/spheroid is administered to a joint adapted for implantation and for cartilage regeneration. In another application, the living cell sheet/spheroid is administered, preferably via percutaneous injection, to an infarcted cardiac tissue as a novel therapy for treating myocardial infarction.

### CROSS-REFERENCE TO RELATED APPLICATIONS

The application is a continuation-in-part of U.S. patent application Ser. No. 11/256,729, filed Oct. 24, 2005. This application also claims priority benefits of provisional patent application Ser. No. 60/861,157, filed Nov. 27, 2006, and Ser. No. 60/906,690, filed Mar. 13, 2007, the entire contents of all are incorporated herein by reference.

### FIELD OF THE INVENTION

The present invention is related to living cell packets in sheets, spheroids or other configurations for tissue reconstructions and regeneration, more particularly; the invention is related to a medical device having a sheet derived from a thermoreversible hydrogel for harvesting living cells.

### BACKGROUND OF THE INVENTION

Fetal cardiomyocytes or stem cells transplanted into myocardial scar tissue improved heart function. However, low cell numbers remain in place because of washout effects. The transplanted allogenic cells survive for only a short time in the recipient heart because of immunorejection. Autologous cell transplantation would be ideal. The cultured skeletal myoblasts have been successfully isolated, cultured, and transplanted into injured and normal myocardium of the same animal. One of the basic problems with cell therapy in myocardial infarct patients is cell leakage from the implanted site.

Methylcellulose (MC) is a water-soluble polymer derived from cellulose, the most abundant polymer in nature. As a viscosity-enhancing polymer, it thickens a solution without precipitation over a wide pH range. This feature makes it widely useable as a thickener in the food and paint industries. It is recognized as an acceptable food additive by the U.S. Food and Drug Administration. Additionally, the physiological inertness and the storage stability of MC permit its use in cosmetics and pharmaceutical products.

Recently, investigations of hydrogels have focused on functional hydrogels. These functional hydrogels may change their structures as they expose to varying environment, such as temperature, pH, or pressure. MC becomes gels from aqueous solutions upon heating or salt addition (Langmuir 2002; 18:7291, Langmuir 2004; 20:6134). This unique phase-transition behavior of MC makes it as a promising functional hydrogel for various biomedical applications (Biomaterials 2001; 22:1113, Biomacromolecules 2004; 5:1917). Tate et al. studied the use of MC as a thermoresponsive scaffolding material (Biomaterials 2001; 22:1113). In their study, MC solutions were produced to reveal a low viscosity at room temperature and formed a soft gel at 37° C.; thus making MC well suited as an injectable scaffold for the repair of defects in the brain. Additionally, using its thermoresponsive feature, MC was used by our group to harden aqueous alginate as a pH-sensitive based system for the delivery of protein drugs (Biomacromolecules 2004; 5:1917).

It is disclosed herein that a novel application of this thermoresponsive MC hydrogel is blended with distinct salts and coated on tissue culture polystyrene (TCPS) dishes as a living-cell-sheet harvest system. It was reported that a thermoresponsive polymer, poly(N-isopropylacrylamide) (PNIPAAm), is chemically grafted on TCPS dishes to develop a cell-sheet for tissue reconstructions (J. Biomed. Mater. Res. 1993; 27:1243). PNIPAAm is hydrophobic at 37° C. and hydrophilic at 20° C., thus the cultured cells can be harvested as a continuous cell sheet after incubation at 20° C. The harvested cell sheets have been used for various tissue reconstructions, including ocular surfaces, periodontal ligaments, cardiac patches, and bladder augmentations (Materials today 2004; 42). In their method, PNIPAAm is polymerized and concurrently grafted to TCPS dishes by means of irradiation with an electron beam. The whole grafting process is relatively complicated and time-consuming (Tissue Eng. 2005; 11:30).

It is herein disclosed that a simple and inexpensive method is provided by simply pouring aqueous MC solutions blended with distinct salts on TCPS dishes at room temperature (about 20° C.) and subsequently gelled at 37° C. (the MC hydrogel). The gelled coating at 37° C. is then evenly spread with a neutral aqueous collagen at 4° C. The spread aqueous collagen gradually reconstitutes with time and thus forms a thin layer of collagen coated on the MC hydrogel. The physical behavior of the prepared MC hydrogels transitions from the solution to a gel state as a function of temperature.

In the orthopedic field, degenerative arthritis or osteoarthritis is the most frequently encountered disease associated with cartilage damage. Almost every joint in the body, such as the knee, the hip, the shoulder, and even the wrist, is affected. The pathogenesis of this disease is the degeneration of hyaline articular cartilage. The hyaline cartilage of the joint becomes deformed, fibrillated, and eventually excavated. If the degenerated cartilage could somehow be regenerated, most patients would be able to enjoy their lives without debilitating pain.

U.S. Patent Application publication no. 2005/0074481, published on Apr. 7, 2005, entire contents of which are incorporated herein by reference, discloses an implantable device for facilitating the healing of voids in bone, cartilage and soft tissue, comprising a polyelectrolytic complex region joined with a subchondral bone region. The polyelectrolytic complex region enhances the environment for chondrocytes to grow articular cartilage; while the subchondral bone region enhances the environment for cells which migrate into that region's macrostructure and which differentiate into osteoblasts.

U.S. Patent Application publication no. 2005/0159820, published on Jul. 21, 2005, entire contents of which are incorporated herein by reference, discloses a member for articular cartilage regeneration being characterized in that the member comprises a hydroxyapatite porous element having a number of pores distributed therein, substantially all of the pores being three-dimensionally communicated to each other through open portions.

An exemplary articular cartilage repairing means that can be used in a method of the invention is described in U.S. Pat. No. 6,835,377 B2, which discloses mesenchymal stem cells for articular cartilage repair combined with a controlled-resorption biodegradable matrix, preferably collagen-based products. These mesenchymal stem cell-matrix implants initiate tissue formation, and maintain and stabilize the articular defect during the repair process. In addition to gels, the types of biomatrix materials that may be used include sponges, foams or porous fabrics that form a three-dimensional scaffold for the support of mesenchymal stem cells. These materials may be composed of collagen, gelatin, hyaluronan or derivatives thereof, may consist of synthetic polymers, or may consist of composites of several different materials. The different matrix configurations and collagen formulations will depend on the nature of the cartilage defect, and include those for both open surgical and arthroscopic procedures.

Human mesenchymal stem cell technology provides not only multiple opportunities to regenerate cartilage, but other mesenchymal tissue as well, including bone, muscle, tendon, marrow stroma and dermis. The regeneration of cartilage and other injured or diseased tissue is achieved by administration of an optimal number of human mesenchymal stem cells to the repair site in an appropriate biomatrix delivery device, without the need for a second surgical site to harvest normal tissue grafts. However, cells without a colony or confluence arrangement usually fails to sustain the proliferation and stability.

Clearly, there remains a need to develop a system and methods whereby living cells on a sheet can be delivered to a deficiency or defect site for treating bone or joint defect in a patient. In view of the foregoing, an object of this invention is to provide a novel method, using a thermoreversible MC/PBS/Collagen hydrogel coated on the TCPS dish, for harvesting a living cell sheet with ECM. The coated hydrogel system is reusable and can be used for culturing a multi-layer cell sheet. The obtained living cell sheets are useful for tissue reconstructions and cell separation.

### SUMMARY OF THE INVENTION

Some aspects of the invention relate to a novel yet simple method, using a thermoreversible hydrogel system that is coated on tissue culture polystyrene (TCPS) dishes, to provide means for harvesting living cell sheets. The hydrogel system is prepared by simply pouring aqueous methylcellulose (MC) solutions blended with distinct salts on TCPS dishes at 20° C. In one embodiment, aqueous MC compositions form a gel at 37° C. for the application of cell cultures. In one embodiment, the hydrogel coating composed of 8% MC blended with 10 g/L PBS (the MC/PBS hydrogel, with a gelation temperature of about 25° C.) stayed intact throughout the entire course of cell culture.

Some aspects of the invention relate to cell attachments comprising evenly spreading the MC/PBS hydrogel at 37° C. with a neutral aqueous collagen at 4° C. The spread aqueous collagen gradually reconstitutes with time and thus forms a thin layer of collagen (the MC/PBS/Collagen hydrogel). After cells reaching confluence, a continuous monolayer cell sheet forms on the surface of the MC/PBS/Collagen hydrogel. When the grown cell sheet is placed outside of the incubator at 20° C., it detaches gradually from the surface of the thermoreversible hydrogel spontaneously, in absence of any enzymes.

Some aspects of the invention relate to a method of preparing a living cell sheet comprising: coating a thermoreversible hydrogel on a tissue culture dish, wherein the hydrogel comprises methylcellulose, phosphate buffered saline, and optionally collagen; loading target living cells into the dish; incubating the dish for a predetermined duration; and removing the sheet from the dish. In one embodiment, the living cells comprise regenerative cells, such as stem cells, mesenchymal stem cells, adult multipotent cells, and the like.

Some aspects of the invention relate to a method of preparing a 3-D living cell construct comprising: coating a thermoreversible hydrogel on a 3-D scaffold support element, wherein the hydrogel comprises methylcellulose, phosphate buffered saline, and collagen; loading target living cells onto the support element; and incubating the support element for a predetermined duration. In one embodiment, the method further comprises a step of removing the construct from the support element.

The results obtained in the MTT assay demonstrate that the cells cultured on the surface of the MC/PBS/Collagen hydrogel had better cell activities than those cultured on an uncoated TCPS dish. After harvesting the detached cell sheet, the remained viscous hydrogel system is reusable. Additionally, the developed hydrogel system is used for culturing a multi-layer cell sheet. The obtained living cell sheets are candidates for tissue reconstructions or tissue regeneration. In one embodiment, the cells of the invention comprise mesenchymal stem cells, adult multipotent cells, progenitor cells, marrow stromal cells. In a further embodiment, the cells of the invention comprise the intermediate cells, such as osteoblast leading to bone, chondrocyte leading to cartilage, adipocyte leading to adipose, and other cell types leading to connective tissue.

Some aspects of the invention provide a composite medical device or an implant comprising a living cell sheet and a support scaffold having at least two layers, wherein the living cell sheet is sandwiched in between the two layers, wherein at least a portion of the sandwiched two layers are further secured to each other. In one embodiment, the method for securing the two layers is selected from a group consisting of sealing, coupling, stapling, and suturing. Furthermore, the living cell sheet is manufactured by a process comprising: coating a thermoreversible hydrogel on a tissue culture dish, wherein the hydrogel comprises methylcellulose, and phosphate buffered saline; loading target living cells into the dish; incubating the dish for a predetermined duration; and removing the sheet from the dish.

In one embodiment, the support scaffold is biodegradable and the living cell sheet may comprise mesenchymal stem cells. In another embodiment, the medical device or the implant may comprise a wound dressing device, a valvular leaflet, a bioprosthetic tissue valve, a ligament tendon substitute, a tendon substitute, a breast insert for breast tissue regeneration, and the like.

It is one object of the present invention to provide a manufacturing process for the support scaffold, wherein the process comprises: removing cellular material from a nature tissue, wherein porosity of the nature tissue is increased at least 5%, the increase of porosity being adapted for promoting tissue regeneration. In one embodiment, increased porosity is provided by an acellularization process, an acid treatment process, a basic treatment process, or an enzyme treatment process. In another embodiment, the manufacturing process further comprises a step of crosslinking the nature tissue.

Some aspects of the invention provide a method for treating a target tissue, comprising: providing a composite medical device comprising a living cell sheet and a support scaffold having at least two layers, wherein the living cell sheet is sandwiched in between the two layers, and wherein at least a portion of the sandwiched two layers are further secured to each other; delivering the composite medical device to the target tissue; and treating the target tissue by cell proliferation. In one embodiment, the living cell sheet comprises mesenchymal stem cells.

Some aspects of the invention provide a living cell packet in a sheet, spheroid, bundle or other configuration that is broken up to pieces sized and configured for loading in the delivery instrument, wherein each piece comprises a plurality of contiguous cells or cells in confluent appearance. In one embodiment, the process of breaking up into pieces comprises a non-contact segmentation means, such as a laser cutting, focused ultrasonic cutting, or water jet cutting.

Some aspects of the invention provide a method for treating a target tissue, comprising: providing a living cell sheet, wherein the living cell sheet is manufactured by a process comprising coating a thermoreversible hydrogel on a tissue culture dish, wherein the hydrogel comprises methylcellulose, and phosphate buffered saline, loading target living cells into the dish, incubating the dish for a predetermined duration, and removing the sheet from the dish; delivering the living cell sheet to the target tissue; and treating the target tissue by cell proliferation. In one embodiment, the living cell sheet is cut, sized, and configured for loading inside a delivery instrument. In another embodiment, the living cell sheet is a strip sheet that is appropriately loaded inside the lumen of the delivery instrument.

Some aspects of the invention provide a method for treating a joint defect in an animal, comprising administering to the animal stem cells, the stem cells being configured in a living cell sheet. In one embodiment, the living cell sheet is sized and configured to be planar at about 100 microns in size (i.e., equivalent diameter) and about one cell thickness. In another embodiment, the living cell sheet contains about at least 100 cells.

Some aspects of the invention provide a method for treating cartilage defects in a patient, comprising delivering to the patient human cells in a sheet form, wherein the human cell sheet covers or contacts at least a portion of the defects, wherein the human cells are mesenchymal stem cells, marrow stromal cells, or chondrocytes.

Some aspects of the invention provide a method of treating a target lesion in an animal, the method comprising administering stem cells or regenerative cells to the lesion, the cells being configured in at least one living cell bundle in vitro prior to the administering step. In one embodiment, the cells comprise stem cells, myocardiocytes, mesenchymal stem cells or adult multipotent cells.

One aspect of the invention provides a method of treating a target lesion comprising administering stem cells or regenerative cells in at least one living cell bundle configuration, wherein the cell bundle further comprises endogenous extracellular matrices (ECM) for administering into the lesion. In one preferred embodiment, the cell bundle is sized to entrap into interstices of the lesion adapted for offering a favorable ECM environment to retain the administered stem cells or regenerative cells, wherein the cell bundle is in a size range of about 50 mg to 400 mg, preferably in a size range of about 100 mg to 300 mg.

One aspect of the invention provides a method of treating a target lesion comprising administering stem cells or regenerative cells in at least one living cell bundle configuration, wherein the cell bundle further comprises the stem cells or regenerative cells in a contiguous manner or in a confluent appearance.

One aspect of the invention provides a method of treating a target lesion comprising administering stem cells or regenerative cells in at least one living cell bundle configuration, wherein the cell bundle comprises cells in a spheroid configuration or a cell sheet configuration.

One aspect of the invention provides a method of treating a target lesion comprising administering stem cells or regenerative cells in at least one living cell bundle configuration, wherein the lesion comprises an infarcted myocardium, the one in a breast, or the one at a joint.

One aspect of the invention provides a method of treating a target lesion comprising administering stem cells or regenerative cells in at least one living cell bundle configuration, wherein the cell bundle comprises a support biomatrix, wherein the support biomatrix comprises hydrogel or biodegradable.

One aspect of the invention provides a method of treating a target lesion comprising administering stem cells or regenerative cells in at least one living cell bundle configuration, wherein the at least one cell bundle is sized and configured for loading in a delivery instrument for administering to the target lesion, wherein the delivery instrument is a catheter with a needle or a syringe with a needle.

### BRIEF DESCRIPTION OF THE DRAWINGS

FIG. 1 shows the DSC thermograms of aqueous methylcellulose solutions (2% by w/v) blended with distinct concentrations of NaCl.

FIG. 2 shows gelation temperatures of aqueous methylcellulose solutions blended with distinct salts: effect of the concentration of salt.

FIG. 3 shows gelation temperatures of aqueous methylcellulose solutions blended with distinct salts: effect of the concentration of methylcellulose.

FIG. 4 shows osmolalities of aqueous methylcellulose solutions blended with distinct salts: effect of the concentration of salt.

FIG. 5 shows osmolalities of aqueous methylcellulose solutions blended with distinct salts: effect of the concentration of methylcellulose.

FIG. 6 shows changes in osmolality of the PBS solution loaded on each studied TCPS dish with time.

FIG. 7 shows photographs of the TCPS dish coated with the MC/PBS hydrogel in sequence: (a) at 20° C.; (b) at 37° C. for 5 min; (c) at 37° C. for 30 min; (d) followed by at 20° C. for 2 min; and (e) followed by at 20° C. for 20 min.

FIG. 8 shows photomicrographs of cells cultured on: (a) an uncoated TCPS dish, 40×; (b) the TCPS dish coated with the 2% MC+1M NaCl hydrogel, 40×; (c) the TCPS dish coated with the 2% MC+0.2M Na₂SO₄ hydrogel, 40×; (d) the TCPS dish coated with the 2% MC+0.2M Na₃PO₄ hydrogel, 40×; and (e) the TCPS dish coated with the MC/PBS (8% MC+10 g/L PBS) hydrogel, 40× and (f) 100×.

FIG. 9 shows schematic illustrations of cells cultured on the TCPS dish coated with the MC/PBS/Collagen hydrogel and detachment of its grown cell sheet.

FIG. 10 shows photomicrographs of cells cultured on: (a) an uncoated TCPS dish; and (b) on the TCPS dish coated with the MC/PBS/Collagen hydrogel for 1, 3, and 7 days, respectively.

FIG. 11 shows photographs of (a) a grown cell sheet on the TCPS dish coated with the MC/PBS/Collagen hydrogel, and (b) its detaching cell sheet. Photomicrographs of the detaching cell sheet with time as (c) to (j).

FIG. 12 shows immunofluorescence images of the cell sheets grown on the TCPS dish coated with the MC/PBS/Collagen hydrogel for: (a) 1 week; and (b) 2 weeks.

FIG. 13 shows immunofluorescence images of: (a) a single-layer cell sheet (CS); (b) a double-layer cell sheet; and (c) a tri-layer cell sheet obtained from the TCPS dish coated with the MC/PBS/Collagen hydrogel; and (d) a tri-layer cell sheet obtained by folding a single-layer cell sheet.

FIG. 14 shows a medical device comprising a support scaffold structure of multiple layers that sandwich a single cell sheet in between two adjacent scaffold layers.

FIG. 15 shows cell sheet preparation and injection methods.

FIG. 16 shows a myocardial regeneration animal model.

FIG. 17 shows in vitro differentiation and labeling of MSC sheets.

FIG. 18 shows the left ventricular (LV) ejection fraction from the animals in the study.

FIG. 19 shows end-systolic ventricular pressure at 12-week post-operatively from the animals in the study.

FIG. 20 shows the Masson's Trichrome stanning of the retrieved hearts from the animals in the animal study.

FIG. 21 shows re-culture of cells in the cell sheets.

FIG. 22 shows MSC spheroids preparation.

FIG. 23 shows the morphologies of MSC spheroids.

FIG. 24(A) show schematic illustrations of the procedures used for the construction of spherically symmetric MSC bundles inherent with the endogenous extracellular matrices for direct intramyocardial injection.

FIG. 24(B) shows the morphology of MSC bundles formed in the plain hydrogel system was highly variable, whereas those generated in the multiwelled hydrogel system were spherically symmetric. Representative photomicrographs of MSC bundles generated in the plain and multiwelled hydrogel systems.

FIG. 25 shows a single cell bundle that was observed in each well in the multiwelled hydrogel system, except for the case with a cell seeding density of 5×10³ cells/well. The size of cell bundles grown in the multiwelled hydrogel system increased significantly with increasing the cell seeding density. Representative photomicrographs of MSC bundles generated in the multiwelled hydrogel system at different cell seeding densities. Scale bars @ 200 μm.

FIG. 26 show the obtained MSC bundles preserved the endogenous extracellular matrices which were constituted of proteins, such as collagen type I and type III, fibronectin, laminin and E-CAM. Representative immunofluorescence images of MSC bundles. Scale bars @ 40 μm.

FIG. 27 show MSC bundles remained intact and the cells in bundles stayed viable, after injection through a needle. Live/dead staining images of 4 optical sections of MSC bundles before and after injection through a needle. Scale bars @ 50 μm.

FIG. 28 show the ability of cell attachment and proliferation of MSC bundles was still preserved, after injection through a needle. The time required for the cells in MSC bundles to attach and proliferate on the surface of a culture plate was shorter than dissociated MSCs. (A) Photomicrographs and (B) immunofluorescence images of dissociated MSCs and MSC bundles after injection through a needle and then seeded on culture dishes taken at distinct time points. Scale bars @ (A) 200 μm and (B) 40 μm.

FIG. 29 shows intramyocardial injection of MSC bundles reduced the infarct size. Photomicrographs of each studied group (stained with Masson's trichrome) retrieved at 12-week postoperatively.

FIG. 30 show most of dissociated MSCs delivered to the heart through a needle were leaked back out of the injection site, while some were found in the myocardial interstices, after intramyocardial injection. In contrast, MSC bundles were able to entrap into the interstices of myocardial tissues and the transplanted cells were mostly localized at the site of injection. Immunofluorescence images of the hearts treated with dissociated MSCs or MSC bundles in the areas of the peri-infarct. Scale bars @ (A-E) 40 μm and (F-J) 20 μm.

### DETAILED DESCRIPTION OF THE EXEMPLARY EMBODIMENTS

The preferred embodiments of the present invention described below relate particularly to preparation of sheets derived from a thermoreversible hydrogel coated on a tissue culture polystyrene dish for harvesting living cells. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

By “living cell packet” is meant herein any configuration or shape (a sheet, a spheroid, a cell packet, a cell pellet, a bundle, or the like) of contiguous living cells arranged and formed from living cells, wherein each living cell packet may comprise tens or more of cells, preferably at least 100 cells, and most preferably at least one thousand cells, in a partially overlapped layers or contiguous manner, preferably in a single layer. In one embodiment, the contiguous living cells are connected through extracellular matrix and have confluent appearance. The living cell packet may be configured in a ball, a pellet, an aggregate, a cylindrical, a wrinkled sheet, a bundle, or any appropriate configuration for delivery and placement at a target tissue site. In a further embodiment, the single cell packet is sized and configured to be planar (the packet or sheet thickness is about one cell size) about 500 microns in average sizes, preferably about 100 microns, and most preferably about 50 microns in average planar sizes.

By “stem cells” is meant herein cells found in all multi-cellular organisms. They retain the ability to renew themselves through mitotic cell division and can differentiate into a diverse range of specialized cell types. The two broad categories of mammalian stem cells are: embryonic stem cells, derived from blastocysts, and adult stem cells, which are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. As stem cells can be grown and transformed into specialized cells with characteristics consistent with cells of various tissues such as muscles or nerves through cell culture, their use in medical therapies has been proposed. In particular, embryonic cell lines, autologous embryonic stem cells generated through therapeutic cloning, and highly plastic adult stem cells from the umbilical cord blood or bone marrow are touted as promising candidates.

Some aspects of the invention provide a method for treating a joint defect in an animal, comprising administering to the animal stem cells, the stem cells being configured in a living cell sheet characterized with a plurality of contiguous cells or substantial amount of contiguous cells. In one embodiment, the living cell sheet or segment is sized and configured to be planar at about 100 microns in size (i.e., equivalent cross-sectional diameter) and about one cell thickness. In another embodiment, the living cell sheet contains about at least 100 contiguous cells.

### Example No. 1

### Gelation of Aqueous MC Solutions

Commercial MC is a heterogeneous polymer consisting of highly substituted zones (hydrophobic zones) and less substituted ones (hydrophilic zones). Aqueous MC solutions undergo a sol-gel reversible transition upon heating or cooling. In the solution state at lower temperatures, MC molecules are hydrated and there is little polymer-polymer interaction other than simple entanglements. As temperature is increased, aqueous MC solutions absorb energy (the endothermic peaks observed in the differential scanning calorimeter, DSC, thermograms discussed later) and gradually lose their water of hydration. Eventually, a polymer-polymer association takes place, due to hydrophobic interactions, causing cloudiness in solution and subsequently forming an infinite gel-network structure (Carbohydr. Polym. 1995; 27:177).

The temperature in forming this gel-network structure, at which the aqueous MC solution does not flow upon inversion of its container, is defined as the gelation temperature herein. Therefore, the gelation temperature of the aqueous MC solution determined by inverting its container should be slightly greater than the onset temperature of the endothermic peak observed in its corresponding DSC thermogram.

It was reported that addition of salts lowers the gelation temperature of the aqueous MC solution (Langmuir 2002; 18:7291). Upon addition of salts, water molecules are placed themselves around the salts, thus reducing the intermolecular hydrogen-bond formations between water molecules and the hydroxyl groups of MC. This can increase the hydrophobic interaction between MC molecules and lead to a decrease in their gelation temperature.

### Example No. 2

### Preparation of Aqueous MC Solutions

MC (with a viscosity of 3,000-5,500 cps for a 2% by w/v aqueous solution at 20° C.) was obtained from Fluka (64630 Methocel® MC, Buchs, Switzerland). Aqueous MC solutions in different concentrations (1%, 2%, 3%, or 4% by w/v) were prepared by dispersing the weighed MC powders in heated water with the addition of distinct salts (NaCl, Na₂SO₄, Na₃PO₄) or in phosphate buffered saline (PBS) in varying concentrations at 50° C. The osmolalities of the prepared aqueous MC solutions were then measured using an osmometer (Model 3300, Advanced Instruments, Inc., Norwood, Mass., USA).

### Example No. 3

### Gelation Temperatures of Agueous MC Solutions

The physical gelation phenomena of aqueous MC solutions with temperature were visually observed and measured by a DSC (Pyris Diamond, Perkin Elmer, Shelton, Conn., USA). Aqueous MC solutions blended with distinct salts (2 ml samples) were exposed to elevating temperatures via a standard hot-water bath. Behavior was recorded at intervals of approximately 0.5° C. over the range of 20-70° C. The heating rate between measurements was approximately 0.5° C./min. At each temperature interval, the solutions/gels were allowed to equilibrate for 30 min. A “gel” criterion was defined as the temperature at which the solution did not flow upon inversion of the container. A DSC was used to determine the transition temperatures of the prepared aqueous MC solutions heating from 20 to 90° C. A heating rate of 10° C./min was used for all test samples.

### Example No. 4

### Preparation of the MC-Hydrogel Coated TCPS Dish

The prepared aqueous MC solutions that had a gelation temperature below 37° C. were used to coat TCPS dishes (Falcon® 3653, diameter 35 mm, Becton Dickinson Labware, Franklin Lakes, N.J., USA). A 45011 of test MC solutions was poured into the center of each TCPS dish at room temperature (about 20° C.). A thin transparent layer of the poured solution was evenly distributed on the TCPS dish. Subsequently, the TCPS dish was pre-incubated at 37° C. for 1 hour and a gelled opaque layer (the MC hydrogel) was formed on the dish. To evaluate whether the salts blended in the MC hydrogel would leach out with time, the coated TCPS dish was loaded with a pre-warmed PBS at 37° C. (2 ml, with an osmolality of 280±10 mOsm/kg). The osmolality of the loaded PBS solution was monitored with time. An uncoated TCPS dish loaded with the same PBS was used as a control.

For the system further coated with collagen, a 0.5 mg/ml aqueous type I collagen (bovine dermis collagen, Sigma Chemical Co., St. Louis, Mo., USA), adjusted to pH 7.4 by dialysis against PBS at 4° C., was evenly spread onto the aforementioned TCPS dish coated with the MC hydrogel at 37° C.

### Example No. 5

### Cell Culture

HFF (human foreskin fibroblasts) were cultured in Dulbecco's modified Eagle's Minimal Essential Medium (12800 Gibco, Grand Island, N.Y., USA) supplemented with 10% fetal bovine serum (JRH, Brooklyn, Australia) and 0.25% penicillin-streptomycin (15070 Gibco, Grand Island, N.Y., USA) in the TCPS dish of Example No. 4. The cells were maintained at 37° C. with 5% CO₂ and the cultured media were changed 3 times a week until ready for use. In one embodiment, some appropriate growth factors may be added into the culture media, wherein the growth factor may be selected from the group consisting of VEGF (vascular endothelial growth factor), VEGF 2, bFGF (basic fibroblast growth factor), aFGF (acidic fibroblast growth factor), VEGF121, VEGF165, VEGF189, VEGF206, PDGF (platelet derived growth factor), PDAF (platelet derived angiogenesis factor), TGF-β (transforming growth factor-β1, β2, β3 and the like), PDEGF (platelet derived epithelial growth factor), PDWHF (platelet derived wound healing factor), insulin-like growth factor, epidermal growth factor, hepatocytic growth factor, and combinations thereof. After reaching confluence, cells were isolated from culture dishes with a 0.05% trypsin and then seeded uniformly on the coated TCPS dishes at a density of 4×10⁴ cells/cm² at 37° C. Cell attachment and growth were observed daily using a microscope. An uncoated TCPS dish was used as a control. Cell viability was assessed by the MTT [3-(4,5-dimethylthiazol-yl)-2,5-diphenyltetrazolium bromide, Sigma] assay. Details of the methodology used in the MTT assay were previously described (J. Biomed. Mater. Res. 2002; 61:360).

### Example No. 6

### Detachment of Cell Sheets

Cells grown on the dishes for 1 or 2 weeks (with media changes 3 times per week) were taken out from the incubator with media present. The dishes were then allowed to cool at approximately 20° C. Changes in morphology of cell sheets on the dishes with time were photographed every 5 seconds for up to 15 min.

### Example No. 7

### Immunofluorescence Staining

Monoclonal mouse anti-collagen type I (1:150, ICN Biomedicals, Inc., Aurora, Ohio, USA) and type III (1:200, Chemicon International Inc., Temecula, Calif., USA) antibodies were used for localizing type I and type III collagen secreted by HFF, respectively. A Cy5-conjugated affinity-purified goat anti-mouse IgG+IgM (H+L) (1.5 mg/ml, Jackson ImmunoResearch Laboratories, Inc., PA, USA) was used as the secondary antibody for labeling the monoclonal antibody. Cell sheets grown on the dishes were fixed in 4% phosphate buffered formaldehyde at 37° C. for 10 minutes and then permeabilized with 0.1% Triton X-100 in PBS containing 1% bovine serum albumin (PBS-BSA) and RNase 100 μg/ml. After washing 3 times with PBS-BSA, the cell sheets were exposed to the primary antibody for 60 min at 37° C. The cell sheets were then incubated for another 60 min with the secondary antibody (1:400) at room temperature. Additionally, the cell sheets were co-stained to visualize F-actins and nuclei acids by phalloidin (Oregon Green® 514 phalloidin, Molecular Probes, Inc., Eugene, Oreg., USA) and propidium iodide (PI, P4864, Sigma), respectively.

Subsequently, the stained cell sheets were evenly mounted on the slides and examined with excitations at 488, 543, and 633 nm, respectively, using an inversed confocal laser scanning microscope (TCS SL, Leica, Germany). Superimposed images were performed with an LCS Lite software (version 2.0).

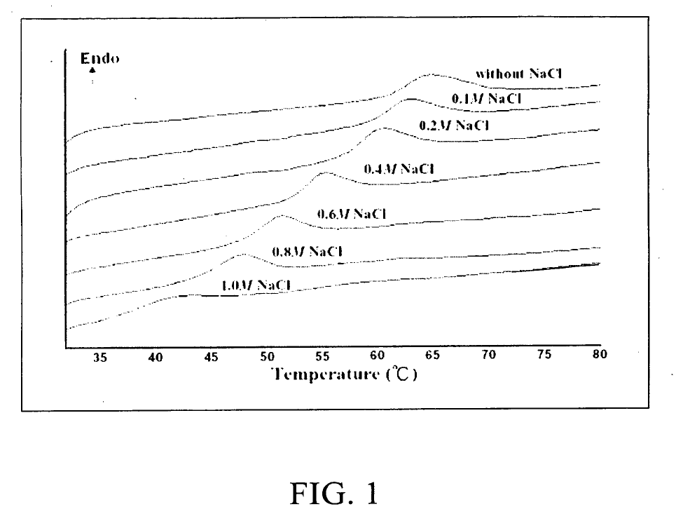

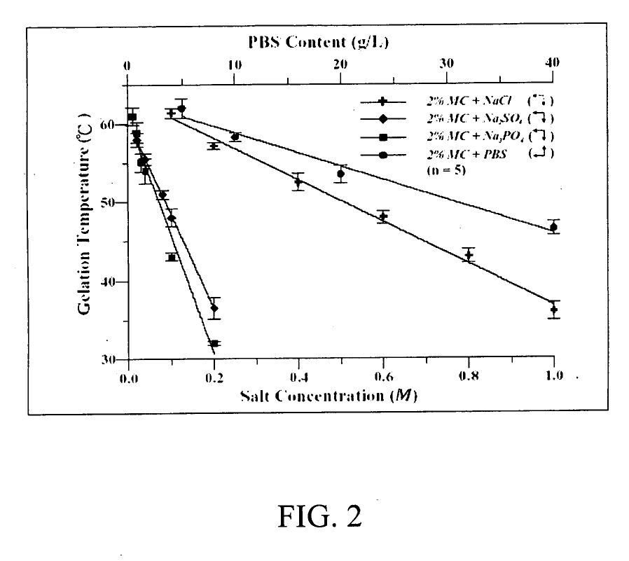

The salts blended in aqueous MC solutions played an important role in their physical sol-gel behavior. Examples of the DSC thermograms of aqueous MC solutions (2% by w/v) blended with distinct concentrations of NaCl are shown in FIG. 1. An endothermic peak was observed for each test sample in the heating process. With increasing the concentration of NaCl, the endothermic peak shifted to the left (p<0.05). This indicates that addition of NaCl in the aqueous MC solution led to its sol-gel transition occur at a lower temperature. Additionally, a higher concentration of NaCl used, a lower temperature in its sol-gel transition was observed. This fact was also observed in the determination of the gelation temperature of each test sample by inverting its container (FIG. 2). As expected, the onset temperatures of the endothermic peaks of aqueous MC solutions observed in the DSC thermograms were lower than their corresponding gelation temperatures obtained by the inversion method, ranged approximately from 1° C. to 3° C. (Table 1).

Similar phenomena were observed when Na₂SO₄, Na₃PO₄, or PBS was blended into aqueous MC solutions (FIG. 2 and Table 1). Normally, an electrolyte (the salt blended) has a greater affinity for water than polymers resulting in removing water of hydration from the polymer and thus dehydrating or ‘salting out’ the polymer. The ability of an electrolyte to salt out a polymer from its solution generally follows the salt order in the lyotropic series. The cations follow the order Li⁺>Na⁺>K⁺>Mg²⁺>Ca²⁺>Ba²⁺, and more common anions follow the order PO₄³⁻>SO₄²⁻>tartrate>Cl⁻>NO₃^{->Br}⁻>I⁻>SCN⁻ (Int. J. Pharm. 1990; 99:233). Accordingly, more water molecules were removed from aqueous MC solutions when Na₂SO₄ or Na₃PO₄ was added in the polymeric hydrogel, resulting in a lower gelation temperature. As shown in FIG. 2 and Table 1, at the same concentration of the salt blended, generally, the gelation temperatures of aqueous MC solutions followed the order Na₃PO₄<Na₂SO₄<NaCl (p<0.05).

Effects of addition of PBS in aqueous MC solutions on the onset temperatures of the endothermic peaks observed in the DSC thermograms and their gelation temperatures were similar to those blended with NaCl, Na₂SO₄, or Na₃PO₄ (FIG. 2 and Table 1). It was reported that the effect of cations on salting-out polymers in solution is less significant than that of anions. Therefore, salting-out MC polymers from aqueous solutions blended with PBS was mainly caused by its constituent anions such as Cl⁻, HPO₄²⁻, or H₂PO⁴⁻.

TABLE 1
The onset temperatures (Tₒₙₛₑₜ) of the endothermic peaks of aqueous
methylcellulose solutions (2% by w/v) blended with distinct salts in
varying concentrations (Conc.) observed in the DSC thermograms and their
gelation temperatures (T_{gelation}) measured by an inversion method (n = 5).
NaCl

| | Conc. (M) | | | | | |
|---|---|---|---|---|---|---|
| | 0.1 | 0.2 | 0.4 | 0.6 | 0.8 | 1.0 |
| Tₒₙₛₑₜ | 59.0 ± 0.8 | 55.6 ± 0.3 | 52.0 ± 0.1 | 47.4 ± 0.3 | 42.3 ± 0.4 | 35.2 ± 0.3 |
| T_{gelation} | 61.4 ± 0.6 | 57.2 ± 0.4 | 52.5 ± 1.1 | 48.0 ± 0.8 | 43.0 ± 0.9 | 36.0 ± 1.1 |

Na₂SO₄

| | Conc. (M) | | | | |
|---|---|---|---|---|---|
| | 0.02 | 0.04 | 0.08 | 0.10 | 0.20 |
| Tₒₙₛₑₜ | 57.3 ± 0.2 | 54.8 ± 0.3 | 50.4 ± 0.4 | 47.4 ± 0.3 | 35.1 ± 0.3 |
| T_{gelation} | 58.0 ± 0.8 | 55.5 ± 0.7 | 51.0 ± 0.5 | 48.0 ± 1.1 | 36.5 ± 1.3 |

Na₃PO₄

| | Conc. (M) | | | | | |
|---|---|---|---|---|---|---|
| | 0.01 | 0.02 | 0.03 | 0.04 | 0.10 | 0.20 |
| Tₒₙₛₑₜ | 60.1 ± 0.5 | 58.4 ± 0.5 | 54.6 ± 0.5 | 53.4 ± 0.3 | 42 ± 0.4 | 30 ± 0.2 |
| T_{gelation} | 61.0 ± 1.1 | 58.9 ± 1.3 | 55.1 ± 1.1 | 54.0 ± 1.7 | 43 ± 1.1 | 32 ± 1.3 |

PBS

Conc. (g/L)

| | 5 | 10 | 20 | 30 |
|---|---|---|---|---|
| Tₒₙₛₑₜ | 57.5 ± 0.2 | 55.1 ± 0.5 | 52.4 ± 0.3 | 44.1 ± 0.2 |
| T_{gelation} | 62.0 ± 1.2 | 58.3 ± 0.5 | 53.5 ± 1.1 | 46.5 ± 0.9 |

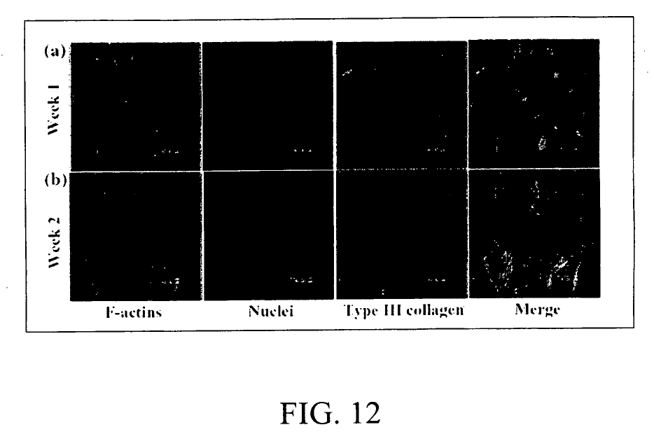

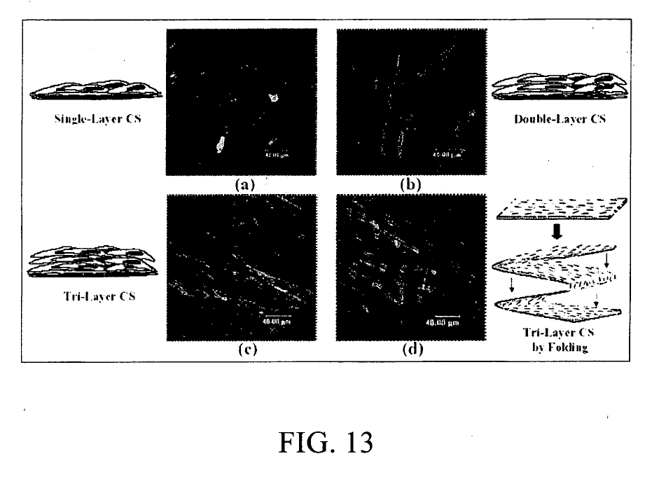

Results of the immunofluorescence images of the cell sheets grown on the MC/PBS/Collagen hydrogel for 1 and 2 weeks are shown in FIGS. 12a and 12b, respectively. As shown, the F-actins and cell nuclei of the cultured cells (HFF) together with the secreted type III collagen were clearly identified. Type I collagen was also found in the study (data not shown). However, the labeled type I collagen may come from the originally coated bovine collagen or that secreted by the cultured cells. These results indicated the cultured cells could secrete their own ECM during culture. On the contrary, the originally coated bovine type I collagen may degrade gradually. It was reported that human skin fibroblasts could secrete collagenase as two proenzyme forms. These enzymes play an essential role in the maintenance of the ECM during tissue development and remodeling (Proc. Natl. Acad. Sci. U.S.A. 1986; 83:3756).

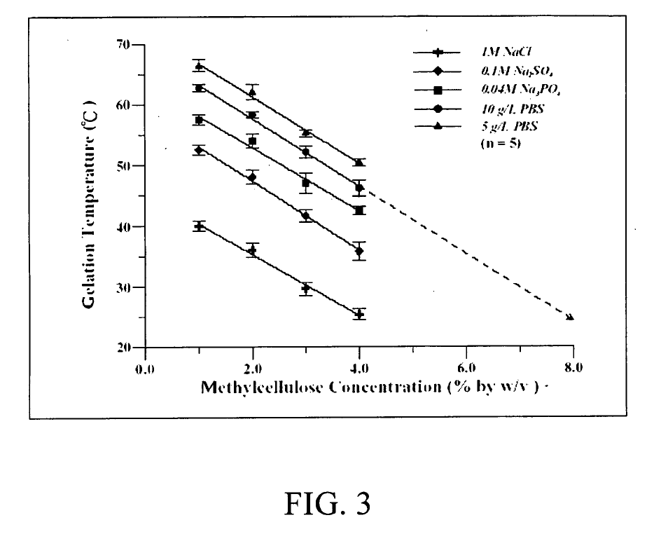

It was found that the concentration of MC in aqueous solution also played a significant role in its physical sol-gel behavior. As shown in FIG. 3, the gelation temperatures of aqueous MC solutions blended with distinct salts decreased approximately linearly with increasing the MC concentration. In the preparation of the aqueous MC solution, it was found that the solution was too viscous to be manipulated with when the MC concentration was greater than about 4% (by w/v). Therefore, no data were available when the concentration of MC was greater than this limit.

For the applications of cell culture, only those aqueous MC compositions that may form a gel (the MC hydrogel) at 37° C. were used to coat the TCPS dishes: 2% MC+1M NaCl; 2% MC+0.2M Na₂SO₄; 2% MC+0.2M Na₃PO₄ (FIG. 2); and 8% MC+10 g/L PBS. For the latter case, a 4% aqueous MC solution blended with 5 g/L PBS was used to coat the TCPS dish and subsequently dried in a laminar flow hood to remove 50% of its moisture content. Thus obtained MC hydrogel had a gelation temperature of about 25° C. (extrapolated from FIG. 3). As shown in FIG. 3, the gelation temperature of a 4% MC solution blended with PBS was significantly greater than 37° C. Additionally, as mentioned above, the aqueous MC solution was too viscous to be manipulated with when its concentration was greater than about 4%. It was observed that this specific aqueous MC solution (8% MC+10 g/L PBS) underwent a sol-gel reversible transition upon heating or cooling at approximately 25° C.

### Example No. 8

### Stability of the Coated MC Hydrogel

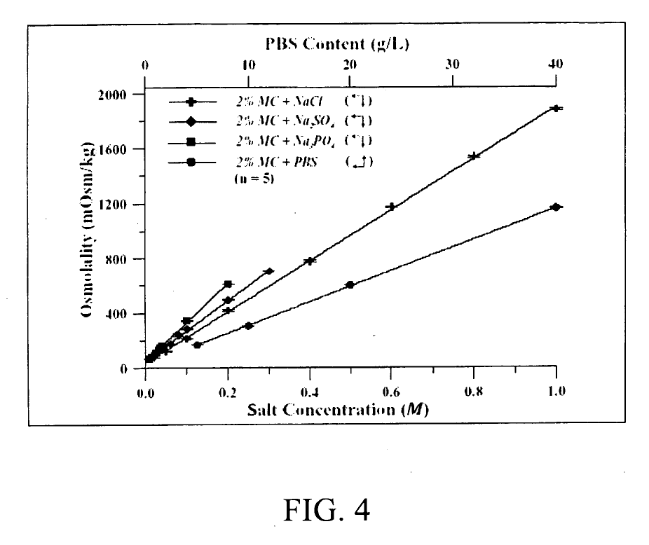

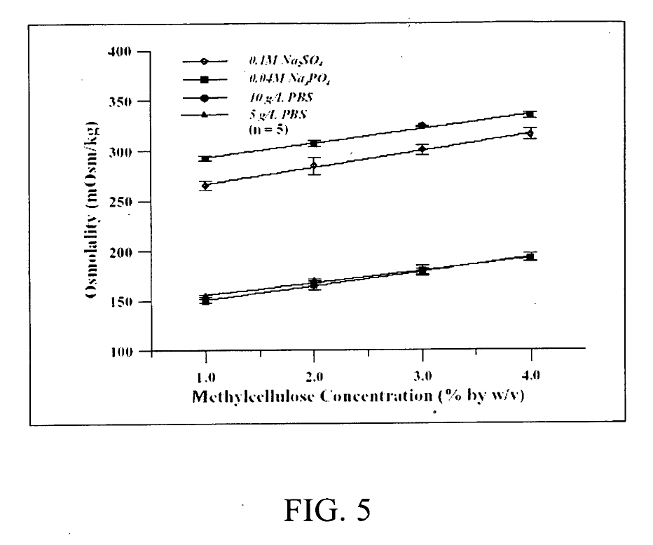

It is suggested that the MC hydrogels coated on TCPS dishes may be swelled and gradually disintegrated when loaded with the cell culture media due to the differences in osmotic pressure between the two. It was found that the osmolalities of aqueous MC solutions, used to prepare the MC hydrogels, increased nearly linearly with increasing the concentrations of the salt blended and MC (FIG. 4 and FIG. 5).

To evaluate the stability of the coated MC hydrogels, a PBS solution (10 g/L) with an osmolality of 280±10 mOsm/kg at 37° C., in simulating that of the cell culture media, was loaded on the coated TCPS dishes. The osmolality of the cell culture media is normally maintained at 290±30 mOsm/kg. An uncoated TCPS dish loaded with the same PBS solution was used as a control. Changes in osmolality of the loaded PBS solution with time were monitored by an osmometer.

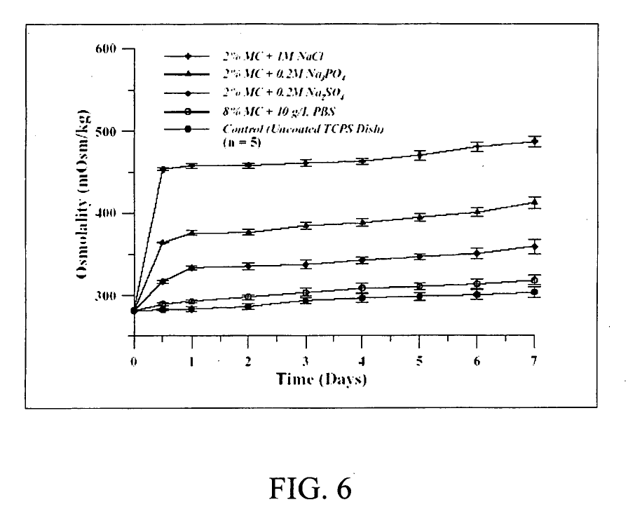

As compared to the uncoated control group, the osmolalities of the loaded PBS solutions increased significantly within 1 day (>325 mOsm/Kg) for the MC hydrogels blended with NaCl, Na₂SO₄, or Na₃PO₄ (p<0.05, FIG. 6). This observation might be attributed to the differences in osmolality between these MC hydrogels (>500 mOsm/kg, FIG. 4) and the originally loaded PBS solutions (about 280 mOsm/kg), and thus caused a significant amount of water from the loaded PBS solutions diffusing into the MC hydrogels. This leads to a significant increase in osmolality for the loaded PBS solutions together with a noticeable swelling and gradual disintegration of the MC hydrogels.

In contrast, the osmotic pressure of the PBS solution (10 g/L) loaded on the MC hydrogel blended with PBS (10 g/L) only increased slightly as compared to the uncoated control group (FIG. 6). Additionally, the MC hydrogel coated on the TCPS dish stayed intact throughout the entire course of the experiment. The aforementioned results indicated that the MC hydrogel blended with PBS (8% by w/v MC+10 g/L PBS) was more suitable for cell cultures than those blended with NaCl, Na₂SO₄, or Na₃PO₄, and thus was chosen for the study (the MC/PBS hydrogel).

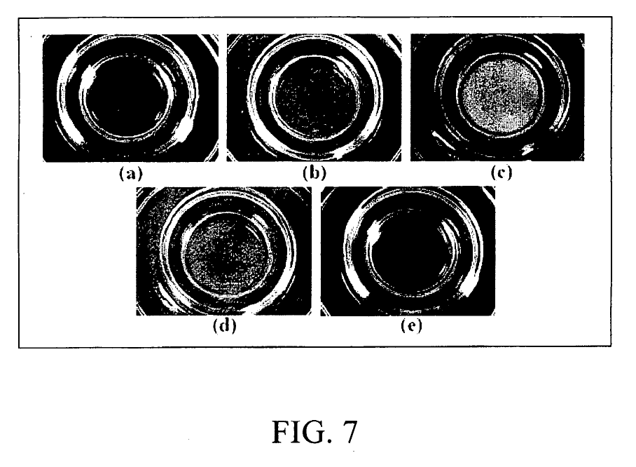

As shown in FIG. 7a, the MC/PBS hydrogel at 20° C. was a clear viscous solution. At 37° C., the clear solution starts to become opaque (FIG. 7b). The transition of sol-gel was continuous with time. At about 30 minutes later, a gel-network structure began to form (FIG. 7c). It was found that this hydrogel was thermoreversible. Back at 20° C., the opaque gel gradually became a clear viscous solution again (FIGS. 7d and 7e).

### Example No. 9

### Cell Culture on the Surface of the MC Hydrogel

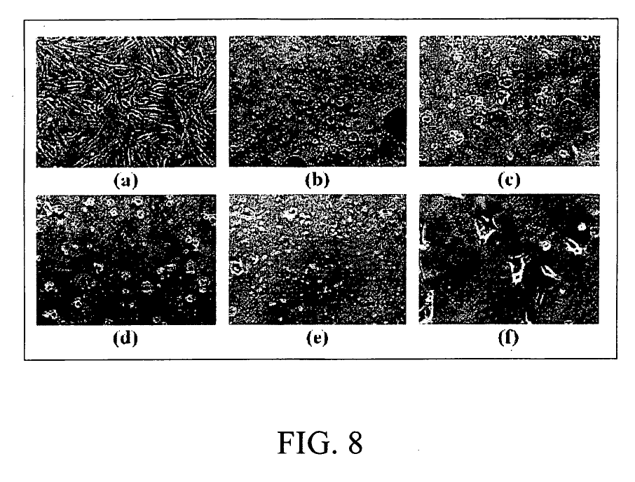

FIGS. 8a to 8f shows photomicrographs of cells (human foreskin fibroblasts, HFF) cultured on the surface of an uncoated TCPS dish (the control group) and those coated with the MC hydrogels blended with distinct slats for 1 day, respectively. As shown, the seeded cells attached very well on the surface of the uncoated TCPS dish (FIG. 8a). However, cells did not attach at all on the surfaces of the MC hydrogels blended with NaCl, Na₂SO₄, or Na₃PO₄ and mainly suspended in the culture media in the form of aggregates (FIGS. 8b-8d). In contrast, a few cells were found to attach on the surface of the MC/PBS hydrogel and the others remained to suspend in the culture media (FIGS. 8e and 8f).

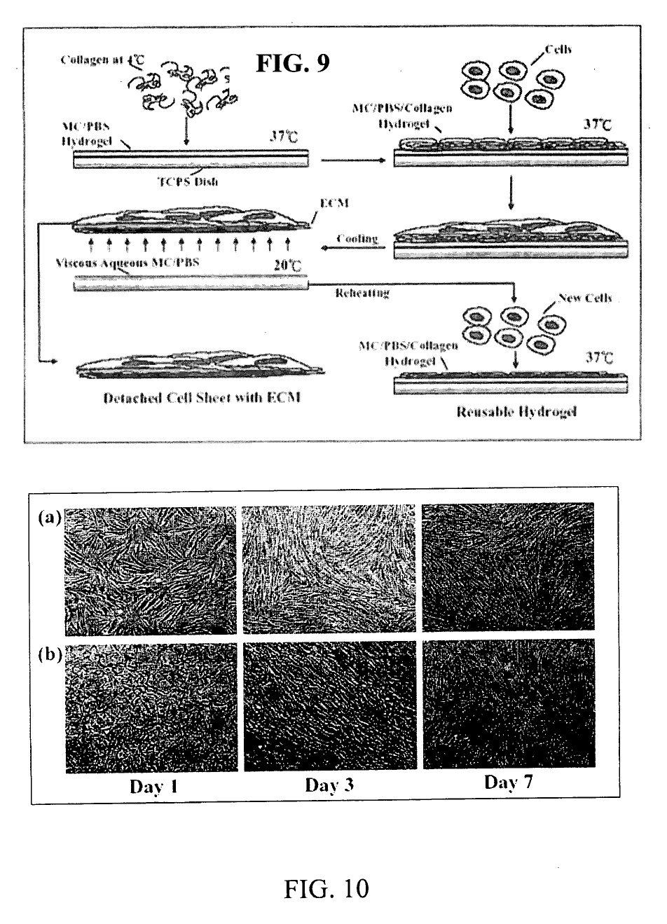

To improve cell attachments, a neutral aqueous bovine type I collagen at 4° C. was evenly spread on the TCPS dish coated with the MC/PBS hydrogel at 37° C. (FIG. 9). It was reported that under the influence of increasing temperature, collagen molecules self-assemble into a gel network. Thermal triggering of collagen gelation was demonstrated at a temperature as low as 20° C. and at a concentration as low as 0.1 mg/ml. Thus a thin layer of bovine type I collagen was formed on the surface of the MC/PBS hydrogel gradually (the MC/PBS/Collagen hydrogel, FIG. 9).

FIGS. 10a to 10i presents photomicrographs of cells cultured on an uncoated TCPS dish and that coated with the MC/PBS/Collagen hydrogel for 1, 3, and 7 days, respectively. Results of their relative-cell-activities of test-to-control evaluated by the MTT assay are shown in Table 2. As shown, after coating with the bovine type I collagen, cell attachments and proliferations were significantly improved as compared to those observed on the surface of the MC/PBS hydrogel (FIGS. 8e and 8f). The results obtained in the MTT assay demonstrated that the cells cultured on the surface of the MC/PBS/Collagen hydrogel had an even better activity than those cultured on the uncoated TCPS dish (p<0.05). Collagen is known to have the capacity to regulate cell behaviors such as adhesion, spreading, proliferation, and migration and thus has been used extensively to enhance cell-material interactions for both in vivo and in vitro applications.

TABLE 2
Results of the relative-cell-activities of test-to-control
obtained in the MTT assay for the cells cultured on an
uncoated TCPS dish (Uncoated Dish) and the TCPS dish coated
with the MC/PBS/Collagen hydrogel (Coated Dish) for 1,
3, and 7 days, respectively (n = 5).

| | Relative Cell Activity^{[a]} | | |
|---|---|---|---|
| | Day 1 | Day 3 | Day 7 |
| Uncoated Dish | 100.0 ± 2.3% | 161.9 ± 9.4% | 203.0 ± 12.3% |
| Coated Dish | 159.1 ± 7.7% | 286.3 ± 13.5% | 339.9 ± 18.7% |

^{[a]}The cell activity of the cells cultured on the uncoated TCPS dish for 1 day was used as a control.

### Example No. 10

### Detachment of Cell Sheets

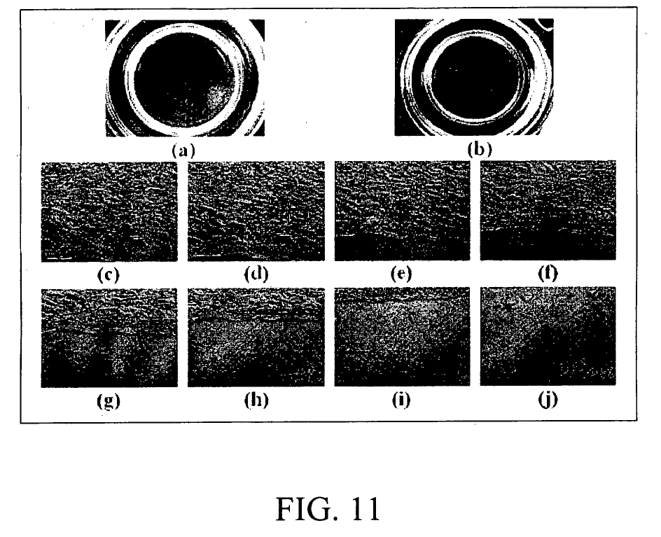

After cells reaching confluence, a continuous monolayer cell sheet formed on the surface of the MC/PBS/Collagen hydrogel (FIGS. 9 and 11a). When the grown cell sheet was placed outside of the incubator at 20° C., it detached gradually from the surface of the thermoreversible hydrogel spontaneously, in absence of any enzymes (e.g., trypsin/EDTA, FIGS. 9 and 11b-11j). It was observed that the grown cell sheet started to detach from its edge at about 2 minutes after cooling at 20° C. Detachment of the entire cell sheet was completed within 20 minutes (or within 10 minutes by shaking the TCPS dish gently with hand). With the same method, a large size of living cell sheet, cultured on a coated 100-mm petri dish, can be readily obtained in our lab and may be utilized in the applications of tissue reconstructions. FIG. 11 shows photographs of (a) a grown cell sheet on the TCPS dish coated with the MC/PBS/Collagen hydrogel and (b) its detaching cell sheet. Photomicrographs of the detaching cell sheet with time (c) to (j).

For most types of cells, and especially for a connective-tissue cell, the opportunities for anchorage and attachment depend on the surrounding matrix, which is usually made by the cell itself. It is known that fibroblasts are dispersed in connective tissue throughout the body, where they secrete an extracellular matrix (ECM) that is rich in type I and/or type III collagen (Molecular Biology of The Cell, 4^{th} ed., Garland Science, New York 2002, Ch. 22). In one embodiment, the detached cell sheet was fixed and immunostained with anti-type I or type III collagen and subsequently co-stained with phalloidin for F-actins and propidium iodide for nuclei acids.

### Example No. 11

### Applications of the Developed Technique

After harvesting the detached cell sheet, the remained viscous MC/PBS hydrogel can be reused subsequent to recoating a thin layer of type I collagen on its surface as described before (FIG. 9). Additionally, a multi-layer cell sheet can be obtained with one of the following two methods. For the first method, a double-layer cell sheet can be obtained by seeding new cells directly on top of the first grown cell sheet (without detaching it from the surface of the MC/PBS/Collagen hydrogel) and then culture until confluence (FIG. 10b). The same procedure can be repeated again to obtain a tri-layer cell sheet (FIG. 10c). The other method is to fold the detached cell sheet into multi layers and reculture it. The folded multi-layer cell sheet would then stick together between layers within 2 days and form an integrated multi-layer cell sheet (FIG. 10d).

Some aspects of the present invention provide a method of preparing a living cell sheet comprising: coating a thermoreversible hydrogel on a tissue culture dish, wherein the hydrogel comprises methylcellulose, and phosphate buffered saline; loading target living cells into the dish; incubating the dish for a predetermined duration; and removing the sheet from the dish. In one embodiment, the hydrogel further comprises collagen. In another embodiment, the hydrogel further comprises at least one growth factor. In another embodiment, the target living cells are mesenchymal stem cells and/or adult multipotent cells.

The aforementioned single-layer or multi-layer cell sheets may be used in the applications of tissue reconstructions or tissue regeneration. Cell sheet engineering is being developed as an alternative approach for tissue engineering. It may have the advantages of eliminating the use of biodegradable scaffolds and maintaining the cultured cell-cell and cell-ECM interactions.

MSC cell sheet may not be easily injected by a needle or catheter into a body (for example, into myocardial tissue, into breast tissue, into an orthopedic space, or the like) of the patient due to its thickness. In one embodiment, each cell sheet (either single-layer or multi-layer sheet) could be broken up to several sub-cellsheets or cut to strips that are sized and configured to be appropriately loaded in a delivery instrument, such as a needle, a syringe, a catheter with a lumen or a cannule. In one embodiment, the cell strip with living cells is loaded into a delivery instrument with its long axis of the cell strip being aligned axially within the axial cavity or lumen of the delivery instrument. This is particularly important to provide therapeutically sufficient amount of MSC to a defect tissue for tissue regeneration by holding the MSC for long enough time on a sub-cellsheet in place at the target tissue site. On the contrary, cells leak or mobilize undesirably under the current cell therapy by injecting cell slurry or cell solution to the target tissue.

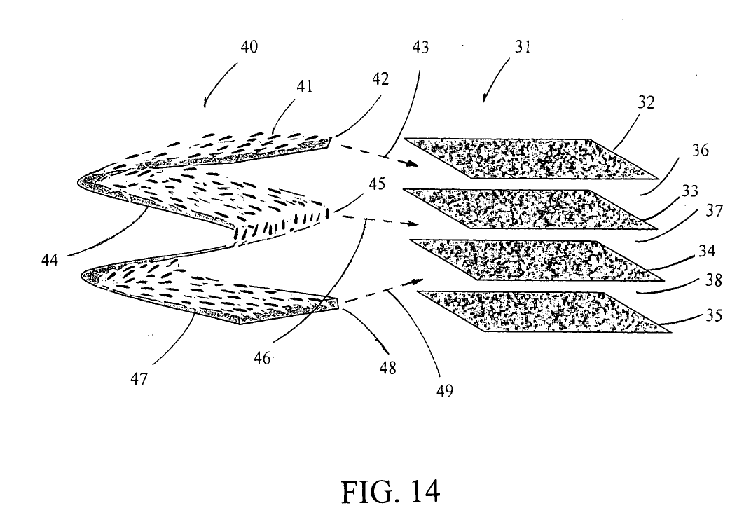

FIG. 14 shows a medical device comprising a support scaffold structure 31 of multiple layers (for example, some discrete layers 32, 33, 34, 35) that sandwich a single cell sheet 40 in between two adjacent scaffold layers, wherein the discrete layers 32, 33, 34, and 35 have a space 36, 37, and 38 between the respective layers as indicated. By way of illustration, a 3-layer living cell sheet 40 comprises layers 41, 44, and 47, whereby each sheet has its sheet edge 42, 45, 48 as indicated, respectively. In preparing a scaffold with living cells in a sandwich manner, the individual sheet edge of the cell sheet 40 is inserted into the space 36, 37, and 38, respectively. For example, the first sheet edge 42 moves toward the space 36 as shown in a dash-lined arrow 43. Similarly, the second sheet edge 45 moves toward the space 37 as shown in a dash-lined arrow 46 and the third sheet edge 48 moves toward the space 38 as shown in a dash-lined arrow 49. In an alternate embodiment, the three layers 41, 44, and 47 are three separate, non-connected living cell sheets.

The sandwiched scaffold may be sealed, secured, coupled, stapled, or sutured at least one edge of the support scaffold structure to enable the composite medical device as a viable integral device or implant. By way of examples, the two adjacent layers with a living cell sheet in between may be sealed with fibrin glue, adhesives, pressure-sensitive adhesives, medical adhesive epoxy system, or cyanoacrylates. In one embodiment, the composite medical device of the invention with loaded living cells is sized and trimmed as a valvular leaflet used in a bioprosthetic tissue valve, as a pericardial patch for tissue regeneration, as a ligament/tendon substitute, as a breast insert for breast tissue regeneration, or as a wound dressing device. The sandwiched composite medical device has the benefits of the support scaffold (for example, an acellular tissue), such as good mechanical property, biocompatibility, and desired porous structure. The sandwiched composite medical device has the benefits of the living cell sheet (for example, multiple cell sheets), such as continuous cell-cell interaction, cell-ECM connection, and multiple cell stack in the composite device. In one embodiment, the support scaffold structure 31 is biodegradable.

In a co-pending patent application Ser. No. 10/408,176, filed Apr. 7, 2003, entitled “Acellular Biological Material Chemically Treated with Genipin”, now U.S. Pat. No. 6,998,418, entire contents of which are incorporated herein by reference, it is disclosed that the support scaffold is manufactured by a process comprising: removing cellular material from a nature tissue, wherein porosity of the nature tissue is increased at least 5%, the increase of porosity being adapted for promoting tissue regeneration. In one embodiment, increased porosity is provided by an acellularization process, an acid treatment process, or a base treatment process. In another embodiment, the manufacturing process for the support scaffold further comprises a step of crosslinking the nature tissue.

In some aspects, the single-layer living cell sheet passes through a laser-assisted cell identification and separation process, wherein a laser light with a cell-specific frequency passes through all cells on the cell sheet in a rotating or programmed manner to identify distinct cells to be preserved (for example, the myocardial stem cells in adipose derived tissue cells). For those non-specific cells or unwanted cells, a laser light with cell destroying energy is emitted to kill those cells. Thereafter, only desired cell type from the single-layer living cell sheet is obtained for cell differentiation and cell regeneration in a recipient. In one embodiment, fluorescence-coded cells or fluorescence light may be used for identifying distinct cells to be preserved to improve the purity of the living cell sheet.

By substituting the tissue culture dish with a 3-dimensional scaffold support element, hydrogel or partially gelled hydrogel of the invention may be loaded or coated onto the support element, followed by loading the target living cells and incubation. In one embodiment, the 3-D scaffold support element is biodegradable or bioresorbable so that the cells-loaded support element serves as an implant for in situ tissue regeneration in a recipient. The biodegradable material for the scaffold support element may be selected from a group consisting of chitosan, collagen, elastin, gelatin, fibrin glue, biological sealant, and combination thereof. The biodegradable material for the scaffold support element may be selected from a group consisting of polylactic acid (PLA), polyglycolic acid (PGA), poly (D,L-lactide-co-glycolide), polycaprolactone, and co-polymers thereof. The biodegradable material for the scaffold support element may be selected from a group consisting of polyhydroxy acids, polyalkanoates, polyanhydrides, polyphosphazenes, polyetheresters, polyesteramides, polyesters, and polyorthoesters.

In one exemplary illustration, hydrogel or partially gelled hydrogel of the invention may be loaded or coated onto the support scaffold element, followed by loading the target living cells and incubation. In one embodiment, the support scaffold element has multiple micropores that are connected to each other and in communication with the exterior surface openings. In another embodiment, the support scaffold element is a pericardial patch tissue, preferably an acellular patch tissue, and most preferably an acellular patch tissue with enlarged pores or increased porosity. U.S. Pat. No. 6,545,042, issued on Apr. 8, 2003, entire contents of which are incorporated herein by reference, discloses a method for promoting autogenous ingrowth of damaged or diseased tissue comprising a step of surgically repairing the damaged or diseased tissue by incorporating a tissue graft, wherein the tissue graft is formed from a segment of connective tissue protein after an acellularization process. In one embodiment, the cell-loaded tissue is sized and trimmed as a valvular leaflet used in a bioprosthetic tissue valve, as a pericardial patch for tissue regeneration, as a ligament/tendon substitute, as a breast insert for breast tissue regeneration, or as a wound dressing device.

Some aspects of the present invention provides a method of preparing a 3-D living cell construct comprising: coating or loading a thermoreversible hydrogel on a 3-D scaffold support element, wherein the hydrogel comprises methylcellulose, phosphate buffered saline, and collagen; loading target living cells onto the support element; incubating the support element for a predetermined duration. In one embodiment, the method further comprises a step of removing the construct from the support element.

Joint Repair or Reconstruction

Inflammation occurs at a joint, for example, associated with arthritis. An example of a joint disease is rheumatoid arthritis (RA) which involves inflammatory changes in the synovial membranes and articular structures as well as muscle atrophy and rarefaction of the bones, most commonly the small joints of the hands. Inflammation and thickening of the joint lining, called the synovium, can cause pain, stiffness, swelling, warmth, and redness. The affected joint may also lose its shape, resulting in loss of normal movement and, if uncontrolled, may cause destruction of the bones, deformity and, eventually, disability. In some individuals, RA can also affect other parts of the body, including the blood, lungs, skin and heart. One aspect of the invention provides delivering a living cell sheet with tissue regeneration capacity for reducing one or more of these adverse symptoms associated with RA.

The knee is a hingelike joint, formed where the thighbone, shinbone, and kneecap meet. The knee is supported by muscles and ligaments and lined with cartilage. Cartilage is a layer of smooth, soft tissue. It covers the ends of the thighbone and shinbone. For reference, U.S. Patent Application publication no. 2006/0029578, describes cartilage in terms of structure, function, development, and pathology in details. The cushioning cartilage can wear away over time. As it does, the knee becomes stiff and painful. Though a knee prosthesis can replace the painful joint, it is always better to regenerate and augment the cartilages with a medical device capable of restoring the cartilage functions by tissue regeneration, particularly the cells that can transform to chondrocytes and eventually to cartilage. One aspect of the invention provides a single cell sheet configured for transformable to chondrocytes at the worn cartilage for cartilage tissue regeneration. By “cartilage” is meant herein including articular cartilage, nose cartilage, ear cartilage, meniscus and avascular cartilage, patellar and spinal disk cartilage, and the like. The delivery means may be via less invasive needle injection or arthroscopic procedures.

A healthy knee joint bends easily. Movement of joints is enhanced by the smooth hyaline cartilage that covers the bone ends, by the synovial membrane that covers the hyaline cartilage and by the synovial fluid located between opposing articulating surfaces. Healthy cartilage absorbs stress and allows the bones to glide freely over each other. Joint fluid lubricates the cartilage surfaces, making movement even easier. A problem knee with worn, roughened cartilage no longer allows the joint to glide freely. Cartilage cracks or wears away due to usage, inflammation or injury. As more cartilage wears away, exposed bones rub together when the knee bends, causing pain. After implanting a living cell sheet, the cartilage is repaired and/or regenerated with new smooth surfaces and the bones can once again glide freely.

An exemplary apparatus for bone marrow collection, transport kit, implant kit and animal models that can be used in a method of the invention is described in U.S. Pat. No. 6,835,377 B2, which is well known to one ordinary skilled in the art and does not constitute a part of the current invention.

For osteoarthritis, rheumatoid arthritis, or fibromyalgia, the problems may occur to any joint, such as a finger joint, knee joint, hip joint, etc. Some aspects of the invention provide at least one living cell sheet as a medical implant for treating cartilage or condyles in arthritis or surface damage of cartilage or condyles. Within six to twelve weeks following implantation, the implant develops into fill thickness cartilage with complete bonding to the subchondral bone.

Some aspects of the invention provide a method for treating cartilage defects in a patient, comprising delivering to the patient human cells in a sheet form, wherein the human cell sheet covers or contacts at least a portion of the defects, wherein the human cells are mesenchymal stem cells, marrow stromal cells, or chondrocytes that are substantially contiguous in the sheet.

It is a further object of the present invention to provide a method for promoting autogenous ingrowth of damaged or diseased tissue selected from the group consisting of bone, ligaments, tendons, muscle and cartilage, the method comprising a step of surgically or interventionally through minimal skin openings, repairing the damaged or diseased tissue by implanting a living cells segment graft, wherein the graft is formed from a segment of a living cells sheet, bundle or cluster, the graft may further be loaded with growth factors, bioactive agents, and the same.

One aspect of the present invention provides a method for forming segments of a living cells sheet using a non-contact, little or no energy cutting means, such as a focused high-pressure liquid-jet knife. Some aspects of the invention provide a process for segmentation of a living cells sheet, comprising: providing a cells sheet; and cutting a segment of the cells sheet with a focused high-pressure liquid-jet, wherein the liquid-jet is supplied with a pressure between about 10 psig and about 10,000 psig, preferably between about 50 psig and about 1,000 psig, wherein the liquid-jet may be operated in a pulsed manner and may be operated with a spot size of about 1 μm to 100 μm in diameter at a sheet contact site, preferably about 10 μm to about 50 μm in diameter at a tissue contact site. One aspect of the invention provides a segment of the living cells sheet produced by the process disclosed herein. In one embodiment, the method and segments are provided with a living cell bundle, the ratio of the contiguous cells portion to the non-contiguous cells portion of the cell bundle or sheet being about 50% or more, preferably about 75% or more, and most preferably, about 90% or more.

Some aspects of the invention provide a process for segmentation of a living cells sheet, comprising: providing a living cells sheet having substantially contiguous cells and extracellular matrix; cutting a segment of the living cells sheet with a laser cutting assembly, wherein the assembly comprises an optic fiber means for delivering desired laser energy to the cells sheet to be segmented.

Some aspects of the invention provide a process for segmentation of a living cells sheet, comprising: providing a cells sheet having substantially contiguous cells and extracellular matrix; and cutting a segment of the living cells sheet with a transducer assembly having a high-intensity focused ultrasound energy source.

Laser Cutting Means

Laser cutting is a technology that uses a laser to cut materials, and is usually used in industrial manufacturing. Laser cutting works by directing the output high power laser, by computer, at the material to be cut. The material then either melts, burns or vaporizes away leaving an edge with a high quality surface finish. However, for cutting a cell sheet to form segments, the laser energy requirement is minimal that barely cuts through the sheet with almost no melting, burning or vaporizing any substantial material. Advantages of laser cutting over mechanical cutting vary according to the situation, but important factors are: lack of physical contact (since there is no cutting edge which can become contaminated by the material or contaminate the material), and to some extent precision (since there is no wear on the laser). There is also a reduced chance of warping the material that is being cut as laser systems have a small heat affected zone. Some materials are also very difficult or impossible to cut by more traditional means.

The most popular lasers for cutting materials are CO₂ and Nd:YAG, though semiconductor lasers are gaining prominence due to greater efficiency. Industrial laser cutters are used to cut flat-sheet material as well as structural and piping materials. Some 6-axis lasers can perform cutting operations on parts that have been pre-formed by casting or machining. Laser cutters usually work much like a milling machine would for working a sheet in that the laser (equivalent to the mill) enters through the side of the sheet and cuts it through the axis of the beam.

There are generally three different types of industrial laser cutting machines. Flying Optics lasers usually feature a stationary X and Y-axis table where the cutting laser moves over the work piece in both of the horizontal dimensions. Flying Optics is popular due to the low cost of stationary tables, and their higher cutting speed limits, since the mass of the optics is much smaller than the mass of the table. Flying optic machines must use some method to take into account the changing beam length from near field (close to resonator) cutting to far field (far away from resonator) cutting. A constant beam length axis is provides the most consistent beam quality over the entire table. Both hybrid and pivot-beam lasers usually involve a table which has the capability of X axis travel. Because of this, the head has to move only in two directions (usually the ones with the shortest runs), thus improving its efficiency, as the path traveled is shorter. Pivot-Beam lasers offer the highest performance per watt and the most reliable cut consistency of the three styles. Hybrid style lasers typically can cut thicker material per watt than other types of laser cutting machines. This is due to the fact that fewer mirrors are required to deliver the laser beam to the cutting head. Each time the laser beam gets reflected by an optic a certain amount of power is lost in the reflective optic.

Pulsed lasers which provide a high power burst of energy for a short period are very effective in some laser cutting processes, particularly for piercing, or when very small holes or very low cutting speeds are required, since if a constant laser beam were used, the heat could reach the point of melting the whole piece being cut.

Focused Ultrasound Energy

It was reported that MR guided focused ultrasound surgery in a non-invasive, outpatient procedure uses high doses of focused ultrasound waves (HIFU) to destroy uterine fibroids. Ultrasound is sound with a frequency greater than the upper limit of human hearing, this limit being approximately 20 kilohertz (20,000 hertz). High-intensity focused ultrasound (HIFU) devices target ultrasound in precise locations for non-invasive surgical treatments. Using diagnostic ultrasound to image a problem area, tumor site or internal trauma injury, a doctor can then point-and-shoot the HIFU transducer and destroy unwanted tissue or cauterize a lesion or blood vessel. With HIFU, instead of dispersing the ultrasound in a fan-like arrangement, which gives you internal images, one can focus the ultrasound like a magnifying glass.

High intensity focused ultrasound is a highly precise medical procedure using high-intensity focused ultrasound to heat and destroy pathogenic tissue rapidly. The ultrasound beam can be focused in these ways: (1) Geometrically, for example with a lens or with a spherically curved transducer; (2) Electronically, by adjusting the relative phases of elements in an array of transducers (a “phased array”). By dynamically adjusting the electronic signals to the elements of a phased array, the beam can be steered to different locations, and aberrations due to tissue structures can be corrected.

As an acoustic wave propagates through the tissue, part of it is absorbed and converted to heat. With focused beams, a very small focus can be achieved deep in tissues. When hot enough, the tissue is thermally coagulated. By focusing at more than one place or by scanning the focus, a volume can be thermally ablated. At high enough acoustic intensities, cavitation (micro bubbles forming and interacting with the ultrasound field) can occur. Micro bubbles produced in the field oscillate and grow (due to factors including rectified diffusion), and eventually implode (inertial or transient cavitation). During inertial cavitation, very high temperatures inside the bubbles occur, and the collapse is associated with a shock wave and jets that can mechanically damage or cut tissue. Cavitation is currently being investigated as a means to enhance HIFU ablation and for other applications. It is contemplated that the laser assembly (58) in FIG. 7 may be replaced with a HIFU assembly for tissue cut purposes.

Some aspects of the invention provide a process for segmentation of a living cell sheet or cell bundle, comprising: providing a cell sheet or bundle having contiguous cells and extracellular matrix; and cutting a segment of the cell sheet or bundle with a transducer assembly having high-intensity focused ultrasound energy source.

Chondrogenesis

This aspect focuses on the identification of molecules regulating mesenchymal stem cells during chondrogenic differentiation, including factors controlling the development of articular hyaline cartilage. To regenerate hyaline cartilage in osteoarthritis patients under a variety of clinical scenarios, it is important to develop a better understanding of the molecules that control the chondrogenic lineage progression of human mesenchymal stem cells. In vitro, it has been possible to culture human mesenchymal stem cells as “pellets” or aggregates under conditions that promote chondrogenesis in serum-free, defined media. This system permits the screening of molecules for chondrogenic potential in vitro. One aspect provides human mesenchymal cells in a single living cell sheet that promotes or enhances chondrogenesis in vivo and in situ.

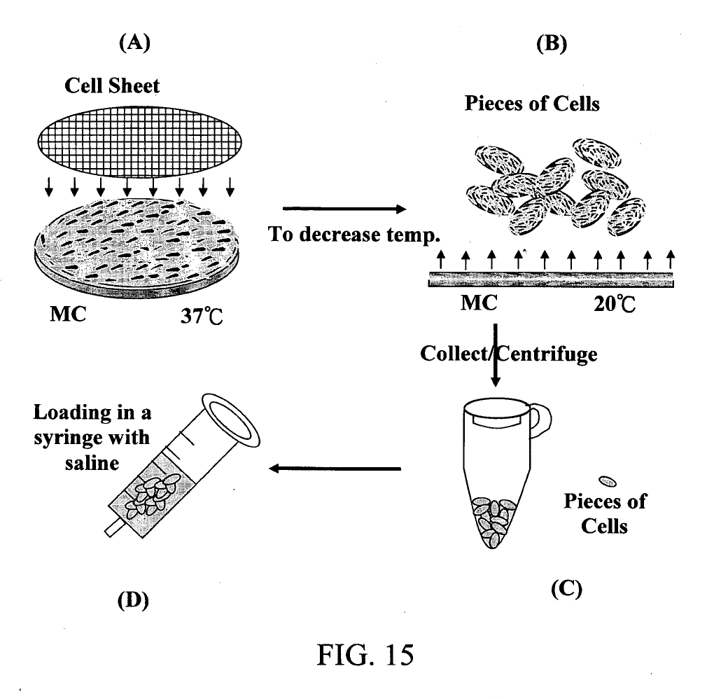

The cell sheet (see FIG. 15) provides biologically acceptable and mechanically stable surface structure suitable for genesis, growth and development of new non-calcified tissue. Other biologically active agents which can be utilized, especially for the reconstruction of articular cartilage, include but are not limited to transforming growth factor beta (TGF-beta) and basic fibroblast growth factor (bFGF).

Molecules that regulate gene expression, such as transcription factors and protein kinases, are useful for monitoring chondrogenesis in vitro, and make it possible to demonstrate, for each sheet or batch of cells, that the mesenchymal stem cells are maintained in an undifferentiated state and, once committed, the mesenchymal stem cell-derived progenitor cells are capable of progressing towards articular chondrocytes. Molecules that are secreted from the developing chondrocytes, such as extracellular matrix components and cytokines, are helpful in monitoring the chondrogenic process in vivo.

An exemplary biomatrix means that can be used in a method of the invention is described in co-pending U.S. patent application Ser. No. 11/287,865, filed Nov. 28, 2005, and entitled “pH sensitive hydrogel and drug delivery system”, which discloses a pharmaceutical composition for treating a joint of a patient, comprising: at least one bioactive agent; and a pH-sensitive hydrogel fluid, wherein the at least one bioactive agent is mixed with the hydrogel fluid, the hydrogel fluid solidifying at a physiological pH of the joint, preferably at a pH range of about 6.0 to 8.0, and most preferably at a pH range of about 7.0 to 7.8. In one embodiment, the bioactive agent is a living cell sheet, preferably a stem cell living cell sheet.

As disclosed, the pH sensitive or temperature sensitive hydrogel fluid may include: (1) a gel formulation that can be applied to osteochondral defects during arthroscopy; (2) an injectable cell-sheet suspension for delivery directly to the synovial space; and (3) a molded mesenchymal stem cell sheet-biomatrix product to re-surface joint surfaces in advanced cases. One aspect of the invention relates to the hydrogel fluid comprising N-alkylated chitosan, wherein the chitosan is optionally crosslinked. Another aspect of the invention relates to the bioactive agent being an anti-inflammatory agent or an anti-infective agent. In one embodiment, the bioactive agent is selected from a group consisting of analgesics/antipyretics, antiasthamatics, antibiotics, antidepressants, antidiabetics, antifungal agents, antihypertensive agents, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, antiplatelet agents and antibacterial agents, antiviral agents, and antimicrobials.

Some aspects of the invention relate to a pharmaceutical composition and a method for treating a joint defect in an animal, comprising administering to the animal stem cells, the stem cells being configured in a living cell sheet. In one embodiment, the method further comprises administering a biomatrix material. In one embodiment, the biomatrix material is a pH-sensitive hydrogel fluid, the hydrogel fluid solidifying at a physiological pH of the joint, preferably at a pH range of about 6.0 to 8.0, and most preferably at a pH-sensitive hydrogel fluid, the hydrogel fluid solidifying at a pH range of about 7.0 to 7.8.

FIGS. 9 and 15 shows cell sheet preparation and injection methods. First, as shown in FIG. 9 and Example No. 9, a cell sheet on MC is prepared by evenly spreading a neutral aqueous bovine type I collagen at 4° C. on the TCPS dish coated with the MC/PBS hydrogel at 37° C., followed by loading target cells onto the collagen suspension. After cells reaching confluence, a continuous monolayer cell sheet formed on the surface of the MC/PBS/Collagen hydrogel (FIGS. 9 and 11a). Second (see FIG. 15A), a cell sheet cutter is used to cut the whole cell sheet into pieces of cells configured for later injection delivery. When the grown cell sheet was placed outside of the incubator at 20° C. (see FIG. 15B), it detached gradually from the surface of the thermoreversible hydrogel spontaneously, in absence of any enzymes. Then pieces of cells at the pre-determined sizes and configuration are collected (see FIG. 15C) and loaded in a syringe (see FIG. 15D) along with saline or biomatrix of the invention for topical injection into a cavity or a joint.

Therapy for Myocardial Infarction

It is known that acute ischemic heart disease is largely caused by complications to myocardial infarction. And myocardial infarction induces acute inflammation, followed by organization and scarring. Clinically, there were several methods available to treat ischemic heart diseases, including thrombolytic agents, PTCA, LVAS, CABG or cardiac transplantation. However, there are limitations to each of the above-mentioned methods, for example: restenosis for PTCA method; durability for LVAS method; and donor shortage for cardiac transplantation method.

Cellular cardiomyoplasty or cell transplantation is an emerging technique for the treatment of myocardial infarction. In the past decade, several cell types implanted into the infarct region have improved ventricular function after a myocardial infarction. However, the cell source might become an issue for a broad clinical application of cardiac cell therapy because expansion of autologous cells could be problematic. Mesenchymal stem cells or MSCs have shown a great potential for cell therapy because these cells possess pluripotent capabilities, proliferate rapidly, induce angiogenesis, and differentiate into myogenic cells. It was demonstrated that MSCs own the potency to differentiate into myogenic cells in the environment of the heart via direct injection or transendocardial injection.

However, the use of trypsin to detach the cells from the culture dish disrupts their microintercellular communication and extracellular matrix, restricts cell survival and growth, and thus appears deleterious to cell therapy. Additionally, other disadvantages include the inability to transplant large numbers of cells and the low viability of transplanted cells. To overcome the aforementioned problems, a novel method for the formation of living cell sheets or spheroids was developed by cultivating cells on a TCPS dish coated with a thermoreversible methylcellulose hydrogel. The prepared aqueous methylcellulose undergoes a sol-gel reversible transition at 32° C. When the cells are confluent in an incubator at 37° C., a continuous monolayer cell sheet is formed on the surface of the coated dish. After moving the culture dish to the room temperature, the formed cell sheet detaches gradually and spontaneously from the surface of the coated dish due to gel-to-solution transition underneath, without being treated with any enzymes. With this novel technique, we are able to cultivate mono- or multi-layer of living cell sheets.

### Example No. 12

### Myocardial Regeneration in Animal Study

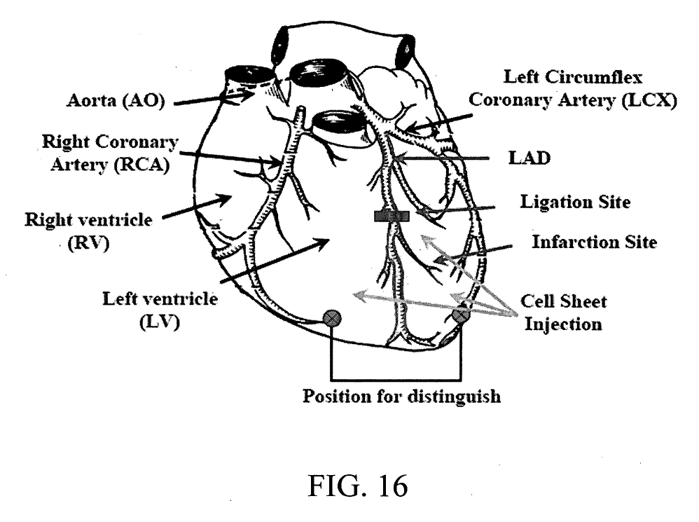

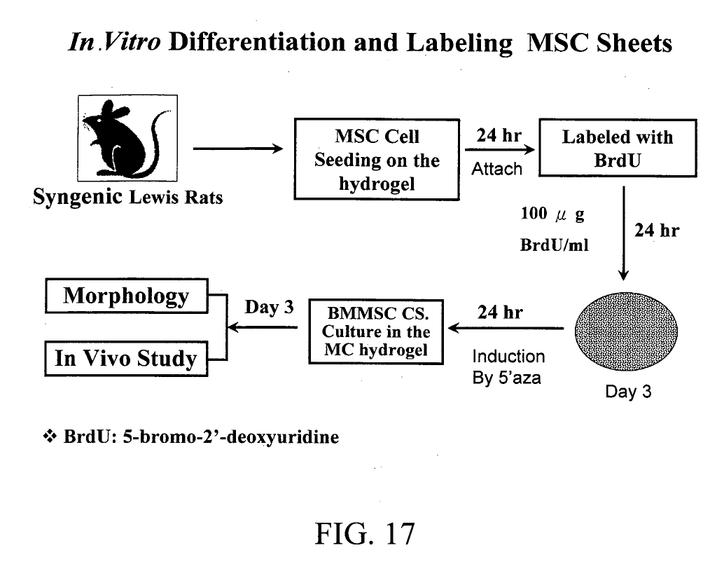

FIG. 16 shows a myocardial tissue regeneration animal model. In one animal study with cell sheet injection, Lewis rats at 350-450 grams were used by ligating between second and third diagonal arteries of the left coronary artery to create an acute myocardial infarction (MI) model. MSC sheet per FIG. 15 was injected to the lesion (at peri-infarct area of the LV wall) of the animal. The control groups included individually dissociated MSC injection, saline injection and sham operations. All animals (n=10 for each group) were followed for 3 months. In the study, MSCs were isolated from syngenic Lewis rats. FIG. 17 shows the isolated MSCs were labeled with BrdU for later identification and subsequently induced by 5-aza towards cardiomyocyte lineage for later in vitro and in vivo studies. The produced MSC cell sheet taken by a confocal laser scanning microscope were imaged. The images were positively stained for troponin-T, a specific marker for cardiomyocyte. This result indicated that the produced MSC cell sheet owns the potency to differentiate to a cardiomyocyte phenotype after 5-aza treatment.

Echocardiography at 4, 8, and 12 weeks postoperatively showed that LV ejection fraction and fraction shortening were significantly improved for the sheet injection group, with a reduced LV end-diastolic dimension, for the MSC sheet injection group as compared with the dissociated MSC and saline groups. In other words, the M-mode echocardiograms of each study group at 3-month postoperatively showed for the group injected with the MSC cell sheets, the contraction of the infracted anterior wall was still preserved, while the contraction of the infarcted anterior wall was limited for the groups injected with saline or the dissociated MSCs.

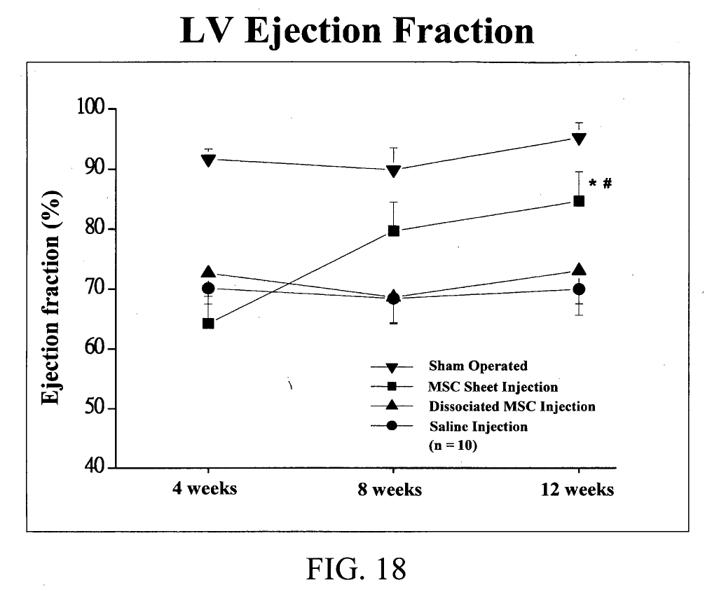

FIG. 18 shows the results of the ejection fraction of left ventricle of the studied animals for each study group. As shown, the ejection fraction of left ventricle for the group injected with the MSC cell sheets continuously improved with time. Such phenomenon was not observed for the groups injected with saline or the dissociated MSCs.

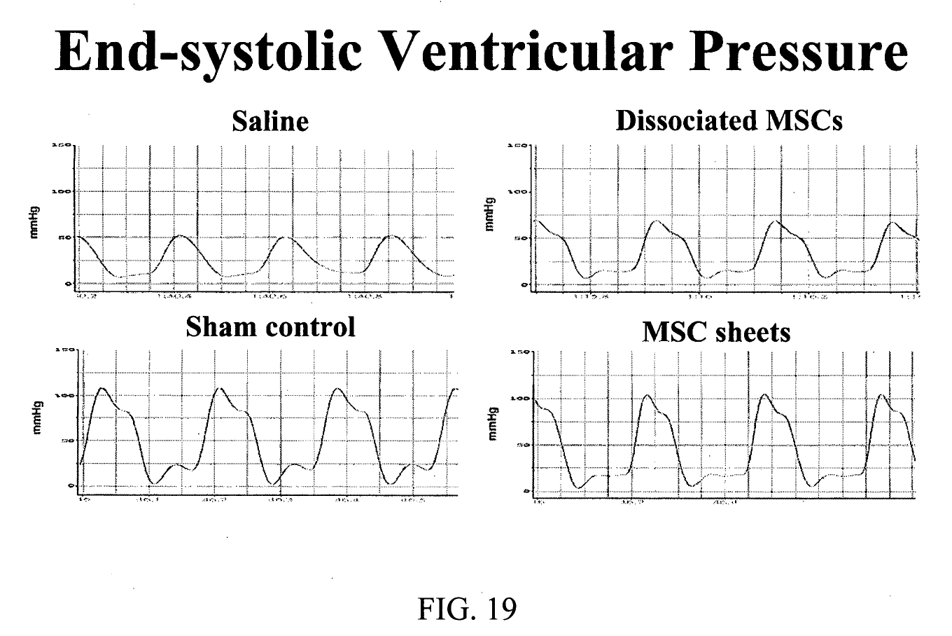

FIG. 19 shows the pressure wave forms observed in the left ventricle for each study group. As shown, the amplitude of the pressure observed for the group injected with the MSC sheet was significantly stronger than the groups injected with saline or the dissociated MSCs. The aforementioned results indicated that the heart function for the group injected with the MSC sheet was significantly better than the groups injected with saline or the dissociated MSCs.

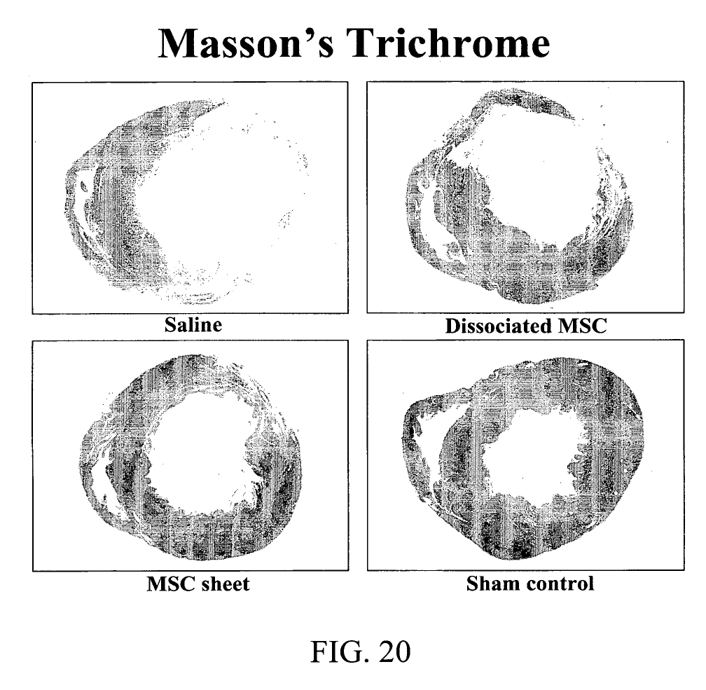

FIG. 20 shows the representative photomicrograph of the retrieved heart for each study group stained with Masson's Trichrome obtained at 3-month postoperatively. As shown, a thinned myocardium with an enlarged left ventricle was observed for the groups injected with saline or the dissociated MSCs. In contrast, the thickness of the infarcted myocardium and the size of the left ventricle were preserved for the group injected with the MSC cell sheets. The Masson's Trichrome histology assessment on the retrieved specimens at 3-month showed numerous cells populated between the infarcted and native myocardium in the MSC sheet injection group.

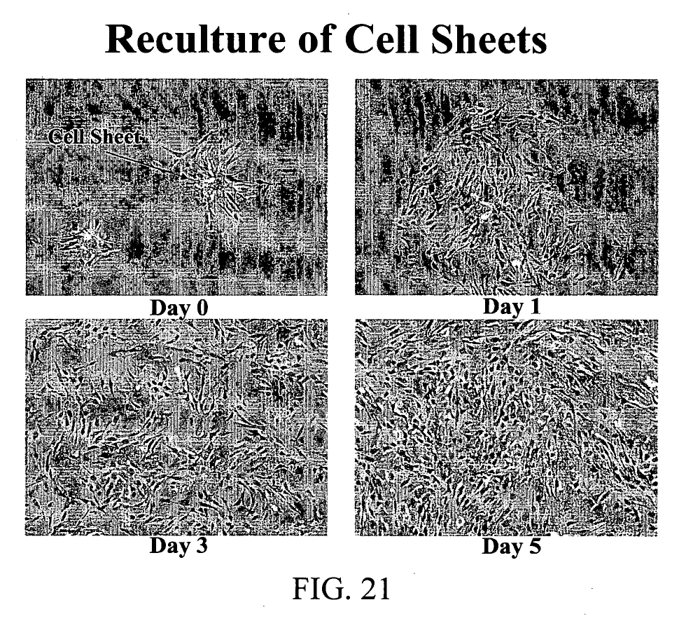

FIG. 21 shows re-culture of cell sheets. The aforementioned results indicated that the injected MSC cell sheets own the potency to differentiate to cardiomyocyte phenotypes. The MSC sheets reversed wall thinning in the scar area and improved cardiac function in rats with acute myocardial infarction. In conclusion, transplantation of MSC sheets is a new therapeutic strategy for myocardial infarction.

### Example No. 13

### Living Cell Spheroid

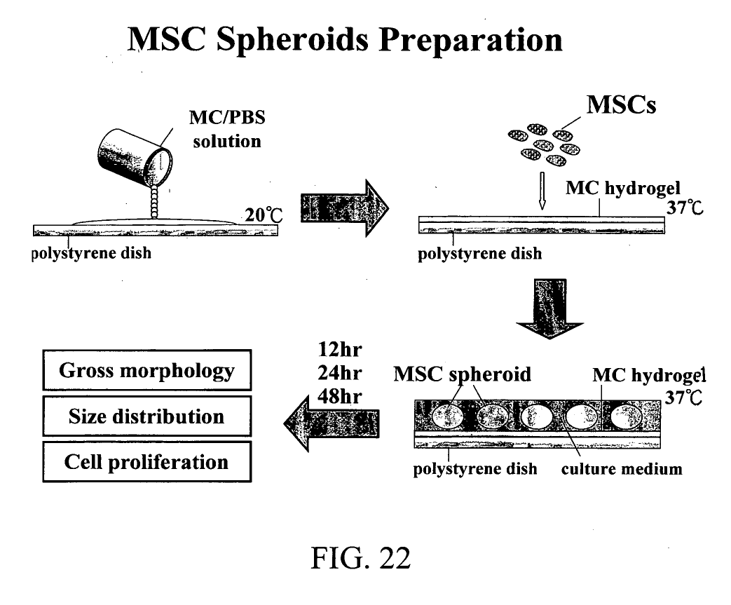

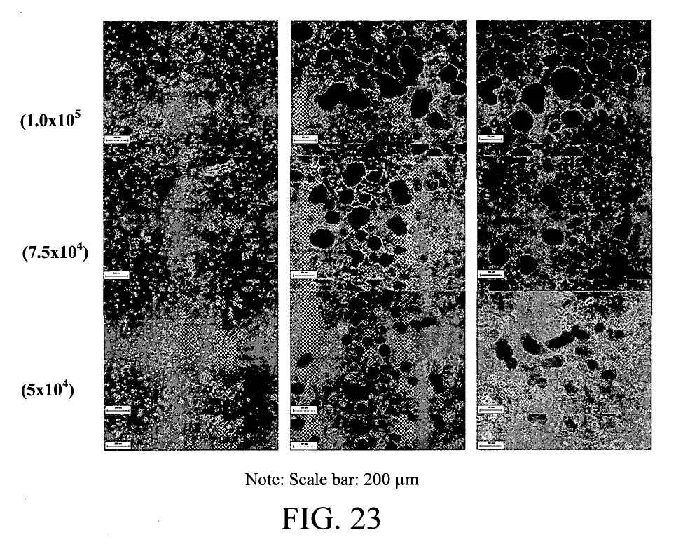

FIG. 22 shows a process for mesenchymal stem cell (MSC) spheroids preparation. The mechanism of cell aggregation involves steps of protein adheres to the bottom of the dish; the cell starts to attach to protein and spread out; when the protein begins to separate from the dish, the cells suspend in the MC solution and agglomerate to form spheroid. In one preferred embodiment, the polyHEMA or chitosan is coated on the dish so to make cells less adherent to the dish for form spheroid. FIG. 23 shows the morphologies of MSC spheroids at different cell densities versus incubation time.

### Example No. 14

### Preparation of the Multiwelled Cell-Bundle Culture System

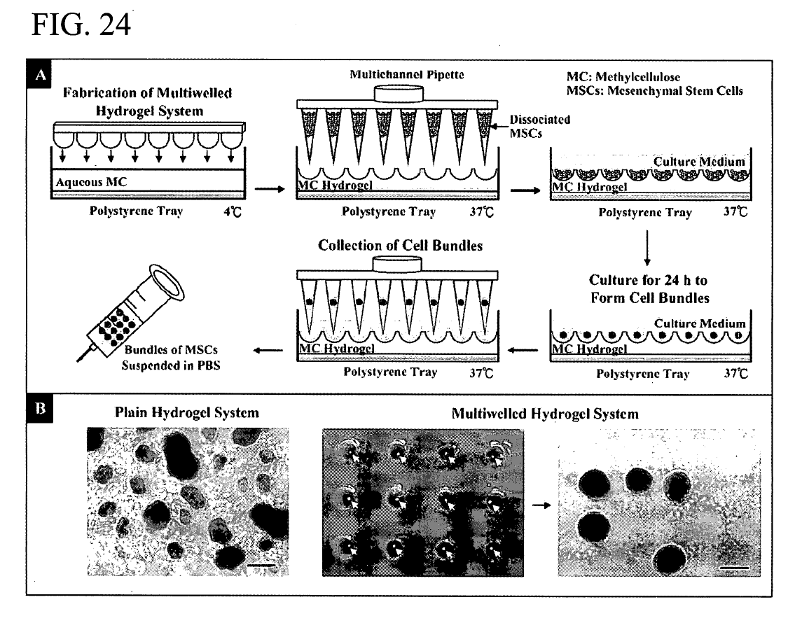

Aqueous MC solutions (12% by w/v) were prepared by dispensing the weighed MC powders (M7027, Sigma-Aldrich, St. Louis, Mo.) in heated water with the addition of phosphate buffered saline (PBS, 5.0 g/l) at 50° C. The prepared MC solution was autoclaved and then kept in a refrigerator at 4° C. for 24 hours. The obtained homogeneous MC solution was poured into a polystyrene tray (Cat. No. 465219, Nalge Nunc International, Rochester, N.Y.) and a 96-well-amplification plate (Cat. No. 230013, Nalge Nunc International) was placed on top of it at 4° C. (FIG. 24A).

Subsequently, the tray was pre-incubated at 37° C. for 2 hours and an opaque gelled layer (3.3±0.1 mm in thickness) with a multiwelled structure (4.0±0.3 mm in diameter) was formed. After gelation, the 96-well-amplification plate was removed and the obtained multiwelled hydrogel system was used to cultivate cell bundles. The plain hydrogel system, without using the 96-well-amplification plate to create the multiwelled structure, was used as a control.

Bone marrow MSCs were isolated from femora and tibia of Lewis rats. The isolated MSCs were spindle-shaped and attached to the culture dish tightly. The DNA-demethylating agent 5-azacytidine (5-Aza, Sigma-Aldrich) was added on the third day and incubated with MSCs for 24 hours. Subsequently, the induced MSCs were labeled for later identification by adding 100 μg/ml 5-bromo-2′-deoxyuridine (BrdU, Sigma-Aldrich) containing media to 50% confluent cultures for 24 hours. After reaching confluence, MSCs were dissociated from culture dishes with a 0.05% trypsin and then seeded in the prepared multiwelled hydrogel system with a multichannel pipette at different cell densities (5.0×10³, 1.0×10⁴, 5.0×10⁴, 1.0×10⁵ or 2.0×10⁵ cells/well) at 37° C. for 24 hours.

### Example No. 15

### Characterization of MSC Bundles

Photomicrographs of cell bundles grown in the multiwelled hydrogel system were taken and their diameters were measured using a computer-based image analysis system (Image-Pro® Plus, Media Cybernetics, Silver Spring, Md., n=10 batches). Examination of the morphology of cell bundles was performed with a scanning electron microscope (SEM, Model S-2300, Hitachi, Tokyo, Japan). The viability of cells in bundles was investigated according to a live/dead assay using calcein AM and ethidium homodimer (Invitrogen, Karlsruhe, Germany). Additionally, cell bundles were trypsinized and subjected to trypan blue dye exclusion to determine total viable cells.

The cell morphology, endogenous ECM and integrative adhesive agents of MSC bundles, before and after injection through a needle, were examined. Briefly, MSC bundles (5×10⁴ cells in total) were resuspended in 3 ml of culture medium, loaded in a syringe, injected through a 27-gauge needle and subsequently seeded onto a 12-well plate (Costar® 3513, Corning, N.Y.). Changes in morphology of MSC bundles on the plates with time were investigated and photographed. Dissociated MSCs (at the same cell density) were used as a control.

Paraformaldehyde-fixed MSC bundles were prepared for immunohistochemistry. The antibodies used were collagen type I (clone I-8H5, MP Biomedical, Solon, Ohio), collagen type III (clone 3G4, Chemicon, Temecula, Calif.), fibronectin (clone IST-9, Abcam, Cambridge, UK), laminin (clone 2E8, Chemicon) and E-CAM (clone G10, Santa Cruz Biotechnology, Santa Cruz, Calif.). Different Alexa Fluor secondary antibodies (Invitrogen) were used to obtain fluorescent colors. MSC bundles were costained to visualize F-actins and nuclei by phalloidin (Alexa Fluor 488 phalloidin) and propidium iodide (PI, Sigma-Aldrich), respectively, and examined using an inverted confocal laser scanning microscope (CLSM, TCS SL, Leica, Wetzlar, Germany).

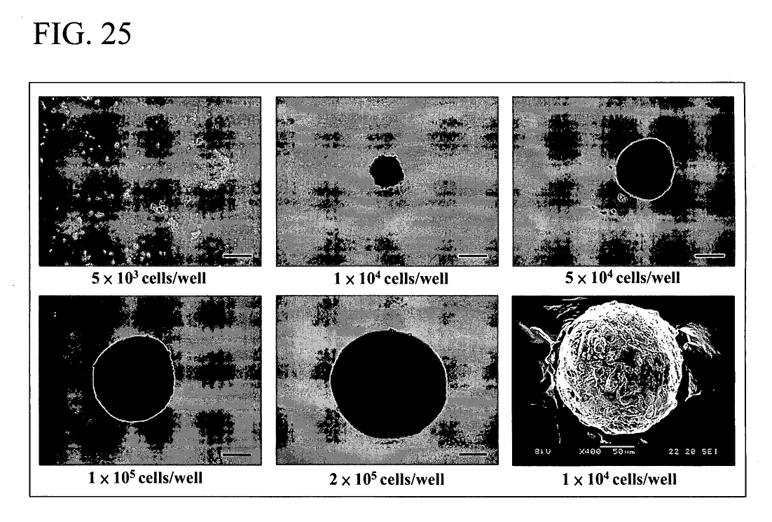

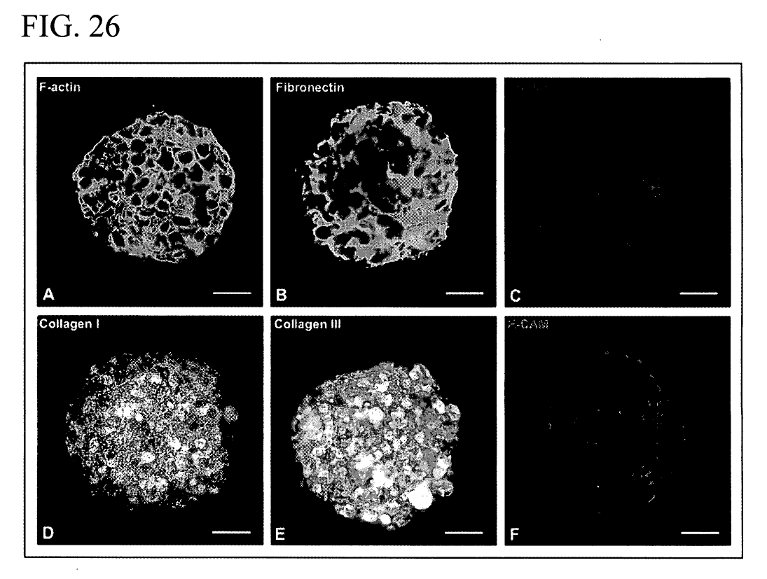

The MSCs (84.5±3.7% BrdU-labelled) seeded in the plain and multiwelled hydrogel systems did not adhere onto substrates; instead, they aggregated and formed cell bundles with time in an appearance of contiguous cells. The morphology of MSC bundles formed in the plain hydrogel system was highly variable, whereas those generated in the multiwelled hydrogel system were spherically symmetric (FIG. 24B) with appearance of contiguous cells. A cell bundle was observed in each well in the multiwelled hydrogel system, except for the case with a cell seeding density of 5×10³ cells/well. The size of cell bundles grown in the multiwelled hydrogel system increased significantly with increasing the cell seeding density (FIG. 25, Table 3). ECM molecules (collagen type I and type III), integrative adhesive agents (fibronectin and laminin) and intercellular junctions (E-CAM) were clearly identified (FIG. 26).

TABLE 3
Sizes of MSC bundles formed in the multiwelled hydrogel
system at different cell densities (n = 7 batches).

| Cell Density (cells/well) | 5.0 × 10³ | 1.0 × 10⁴ | 5.0 × 10⁴ | 1.0 × 10⁵ | 2.0 × 10⁵ |
|---|---|---|---|---|---|
| Mean Diameter (μm) | N/A* | 195 ± 15 | 465 ± 18 | 632 ± 25 | 875 ± 30 |

*Data are not available because the cells seeded at this density did not form a single cell bundle as shown in FIG. 25.

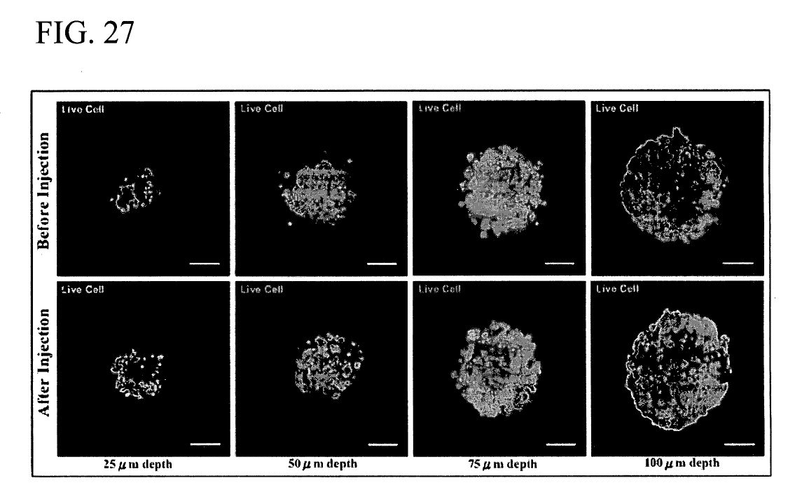

After injection through a 27-gauge needle (inside diameter 400 μm), the MSC bundles formed at a cell seeding density of 1.0×10⁴ cells/well (diameter ˜195 μm) still remained intact. In contrast, the bundles generated at a cell seeding density of 5.0×10⁴ cells/well (diameter ˜465 μm) or beyond often were stuck in the needle and were torn into pieces. Therefore, the cell bundles grown at a cell seeding density of 1.0×10⁴ cells/well were chosen for further studies. Live/dead staining demonstrated that most of the cells in bundles were viable, based on the fluorescence images of 50 optical sections (FIG. 27). The total viable cells before and after injection (9100±85 and 8900±70 cells/bundle, respectively) were found to be comparable, determined by trypan blue dye exclusion.

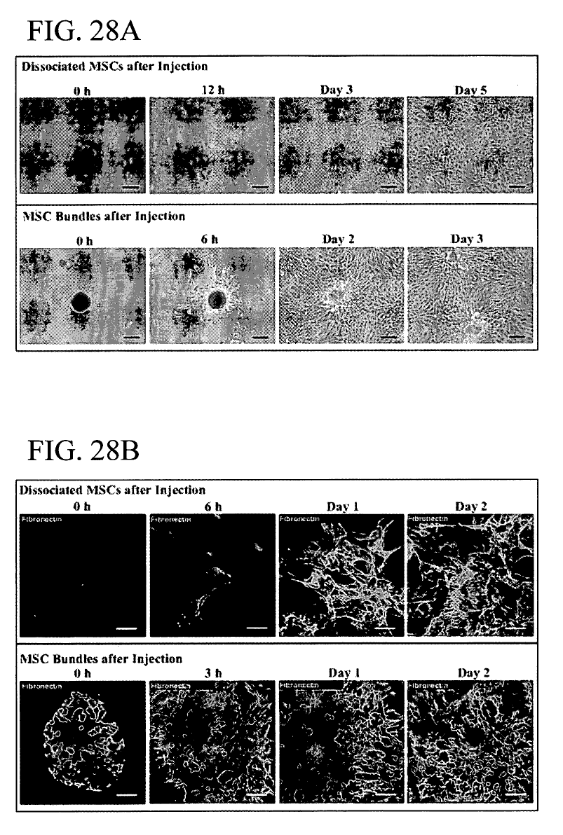

After injection, dissociated MSCs and MSC bundles were individually seeded onto 12-well plates. It took awhile for dissociated MSCs to settle down and spread out on the culture plate (FIG. 28A). Analyses of immunofluorescent images indicated that there was no fibronectin deposited on the plate surface initially. Six hours later, fibronectin was organized into short linear streaks and the cells started to attach to the plate surface (FIG. 28B).

In contrast, MSC bundles adhered to the culture plate shortly after seeding. Subsequently, the cells migrated out of bundles, attached and proliferated on the culture plate (FIG. 28A). A robust fibronectin meshwork inherent with the endogenous ECM was clearly observed in MSC bundles originally; this fibronectin meshwork started to attach to the plate surface within 1 hour and those cells migrated out of bundles continuously produced fibronectin and deposited it onto the plate surface (FIG. 28B). The time required for cell confluence was significantly shorter for the MSC-bundle group (2-3 days) than the dissociated-MSC group (4-5 days, FIG. 28A).

### Example No. 16

### Animal Study with MSC Bundles

The investigation conformed to the Guide for the Care and Use of Laboratory Animals published by the US National Institutes of Health (NIH Publication No. 85-23, revised 1996). Acute myocardial infarction was created in male syngeneic Lewis rats weighing 300-350 grams. After the left coronary artery (LCA) ligation, color changes in the left ventricular (LV) muscle were noticed in all rats. Thirty minutes after myocardial infarction, the rats were randomly divided into four treatment groups: sham (without the LCA ligation); PBS (300 μl); dissociated MSCs (5×10⁵ cells) in PBS; and MSC bundles (5×10⁵ cells in total) in PBS.

An intramyocardial injection of PBS or dissociated MSCs directly into the border zone of the infarct was performed with a 30-gauge needle, while that of MSC bundles was conducted with a 27-gauge needle. Animals were coded so that all measurements were made without knowledge of treatment groups. The study was continued until at least 10 rats survived at least 3 months in each of the 4 coded groups. The overall surgical mortality rate, defined as animal death within 24 hours after surgery, was 6.6% (4 of 60 rats), and the late mortality rate (death between 24 hours and 12 weeks after surgery) was 8.9% [5 of 56 (PBS group, n=3; dissociated-MSC group, n=1; MSC-bundle group, n=1)].

### Example No. 17

### LV Function Assessment by Echocardiography and Catheterization

Echocardiography was performed at 4, 8 and 12 weeks postoperatively for all studied groups. Dimension data were presented as the average of measurement of 5 consecutive beats. The fractional shortening (FS) of LV was calculated as follows:

LVFS(%)=[(LVEDD−LVESD)/LVEDD]×100%

where LVEDD and LVESD corresponded to LV dimensions in end-diastole and end-systole, respectively. Pressure measurements were performed at 12 weeks postoperatively. The aforementioned measurements were conducted by investigators blinded to the experimental conditions. The MSC-bundle group showed a statistically significantly greater LVFS than the dissociated-MSC group at 12 weeks postoperatively. The improvement in LV function for the group treated with MSC bundles was further indicated by a significant increase in LVESP and a decrease in LVEDP when compared with its counterpart treated with dissociated MSCs (Table 4).

TABLE 4
Parameters of LV function and postmortem morphometry.

| | | | |
|---|---|---|---|
| | | Dissociated | MSC |
| Sham | PBS | MSCs | Bundles |

Echocardiographic Data

| | | | | |
|---|---|---|---|---|
| 4 weeks, n value | 11 | 15 | 15 | 15 |
| LVFS (%) | 54.9 ± 2.5 | 32.7 ± 3.9 | 33.3 ± 3.1 | 33.8 ± 4.7 |
| 8 weeks, n value | 11 | 14 | 14 | 15 |
| LVFS (%) | 55.5 ± 3.1 | 29.6 ± 3.8 | 35.3 ± 2.8 | 38.6 ± 6.9^{†} |
| 12 weeks, n value | 11 | 12 | 14 | 14 |
| LVFS (%) | 58.4 ± 4.2 | 25.3 ± 4.1 | 35.4 ± 4.8^{†} | 43.0 ± 5.4*^{†} |

Hemodynamics

| | | | | |
|---|---|---|---|---|
| LVESP (mmHg) | 118.3 ± 8 | 72.5 ± 15 | 85.4 ± 12^{†} | 107.3 ± 11*^{†} |
| LVEDP (mmHg) | 4.5 ± 2 | 15.6 ± 5 | 13.5 ± 4^{†} | 9.5 ± 3*^{†} |

Postmortem analysis

| | | | | |
|---|---|---|---|---|
| Infarct Size (% of LV) | N/A | 38.4 ± 5.2 | 32.1 ± 4.1^{†} | 26.1 ± 4.6*^{†} |
| Infarct Thickness (mm) | N/A | 0.567 ± 0.06 | 0.865 ± 0.05^{†} | 1.15 ± 0.04*^{†} |
| Peri-Infarct Thickness (mm) | N/A | 1.04 ± 0.09 | 1.37 ± 0.13^{†} | 1.85 ± 0.12*^{†} |
| Peri-Infarct Vascular Density | 355 ± 25^{††} | 95 ± 10 | 182 ± 11^{†} | 245 ± 19*^{†} |
| (vessels/mm²) | | | | |

Note:
Values are mean ± SD. LVFS: left ventricular fractional shortening; LVESP: left ventricular end-systolic pressure; LVEDP, left ventricular end-diastolic pressure.
*P < 0.05 vs the dissociated-MSC group;
^{†}P < 0.05 vs the PBS group.
^{††}Vascular density in the normal myocardium.

### Example No. 18

### Histological Examinations

LV myocardium specimens were retrieved at day 1 (n=5 for dissociated-MSC and MSC-bundle groups only) or 12 weeks postoperatively (n≧10 for all studied groups). Specimens used for light microscopy were fixed in 10% phosphate buffered formalin, embedded in paraffin and stained with Masson's trichrome. The stained sections were used to measure and calculate the thickness values of the peri-infarct and infarct areas in each studied group. The infarct size was expressed as the percentage of total LV circumference. Additional sections were stained for factor VIII with an immunohistological technique with a monoclonal anti-factor VIII antibody (DAKO, Carpinteria, Calif.). The vascular density in the peri-infarcted area of all animals was quantified using the above-mentioned image analysis system.

For immunofluorescent staining, after rehydration and microwave antigen retrieval with 0.1 mol/l sodium citrate, sections were incubated at 4° C. for 12 hours with the anti-BrdU antibody resuspended in the dilution buffer. The sections were then double-stained with antibodies against fibronectin, macrophage (CD68, clone ED1, Serotec, Oxford, UK), α-sarcomeric actin (clone 5C5, Serotec, Oxford, UK), factor VIII, α-smooth muscle actin (α-SMA, clone 1A4, DAKO), smooth muscle myosin heavy chain (SMMHC, clone 1G12, Abcam), cleaved caspase-3 (clone 5A1, Cell Signaling Technology, Beverly, Mass.), α-actinin (clone EA 53, Sigma-Aldrich) or the early marker of myocyte development Nk×2.5 (clone N-19, Santa Cruz Biotechnology). The stained sections were counterstained to visualize nuclei by Sytox blue (Invitrogen) or PI. The number of apoptotic cells (or macrophages infiltrated) per field, immunostained for cleaved caspase-3 (or CD 68), was counted and expressed as a percentage of total cells.

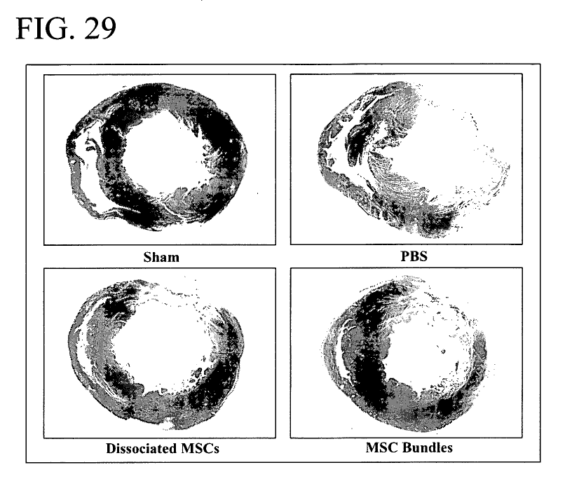

A moderate degree in LV dilation and myocardial fibrosis was observed for the group treated with dissociated MSCs (FIG. 29). In contrast, the group treated with MSC bundles attenuated the enlargement of LV cavity and the development of myocardial fibrosis. The size of the infarct observed in the MSC-bundle group was significantly smaller than in the dissociated-MSC group, while its thickness values and the vascular density were significantly greater (Table 4).

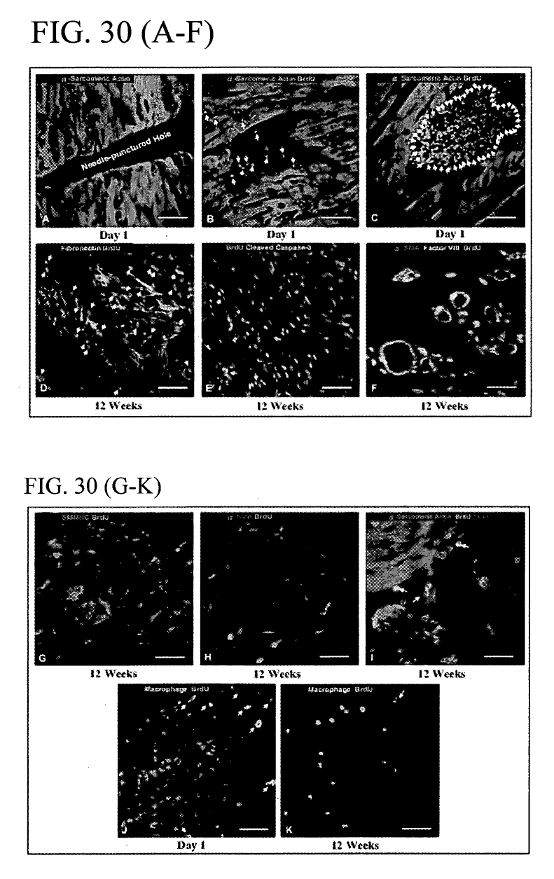

At day 1 after intramyocardial injection, most of dissociated MSCs delivered to the heart through a needle were leaked back out of the injection site, while some were found in the myocardial interstices (FIGS. 30A and 30B). In contrast, MSC bundles were able to entrap into the interstices of myocardial tissues and the transplanted cells were mostly localized at the site of injection (FIG. 30C). At 12 weeks postoperatively, there were still a large number of BrdU-labeled cells adhered to fibronectin retained at the site of injection and there was little detectable cleaved caspase-3 in the MSC-bundle group (<0.5%, FIGS. 30D and 30E); however only a few BrdU-labeled cells were identified in the dissociated-MSC group.

In the MSC-bundle group, some neo-microvessel walls composed of BrdU-labeled endothelial cells (or smooth muscle cells, SMCs) were recognized (FIG. 30F). A significant number of the BrdU-labelled cells were further stained positively for α-SMA or SMMHC, indicating that a substantial portion of the implanted MSCs had been differentiated into myofibroblasts or SMCs (FIGS. 30G and 30H). Also, a few BrdU-labeled cells were stained positively for Nk×2.5 (FIG. 30I), suggesting that a small fraction of the transplanted MSCs had been differentiated into cardiomyocyte-like cells. However, no mature cardiomyocytes α-actinin-positive cells) were identified. Quantification results demonstrated that the percentage of macrophages present at the site of intramuscular injection was 10.5±2.8% at day 1 (FIG. 30J). At 12 weeks postoperatively, the number of macrophages decreased significantly (1.8±0.6%, FIG. 30K).

### Example No. 19

### Assessment of Cell Bundles Application

Typical cell transplantation techniques involve the administration of dissociated cells directly injected into the myocardium; and they do not give the transplanted cells a temporary matrix to which they can attach. In the study, we demonstrated that cell bundles could provide an adequate physical size to entrap into the myocardial interstices and offer a favorable ECM environment to retain the transplanted cells at the sites of injection.

It was shown that the hydrated surface of the MC hydrogel is hydrophilic and neutrally charged. Such kind of culture surface can effectively inhibit the protein adsorption and the attachment of cells onto substrates. Previous work has shown that free-floating MSCs can form multicellular aggregates. Cell adhesion molecules such as integrins and cadherins have been implicated in participating in the process of formation of cell aggregates.

The cell bundles grown in the plain hydrogel system showed a variety of morphologies, as the free-floating MSCs adhered to each other in a random fashion in varying amounts. To overcome this problem, we seeded a fixed amount of cells in each well of the multiwelled hydrogel system so that only the cells within each well could adhere to each other. This technique can produce spherically symmetric cell bundles with a relatively homogeneous size distribution in a short formation time (within 24 hours); factors that are crucial for a better control of cell delivery via intramuscular injection.

The MSC bundles grown at a cell seeding density of 1.0×10⁴ cells/well had a radius of approximately 100 μm; and most of the cells within bundles were viable as indicated by the live/dead staining assay. For the bundles generated at a cell seeding density of 5.0×10⁴ cells/well or beyond (radius>200 μm), the cells deeply embedded inside bundles were difficult to image by CLSM due to the penetration limit of the laser light (˜100 μm from the surface). Dense cellular structures develop hypoxia at distances beyond the diffusion capacity of oxygen (typically ˜200 μm in thickness). Beyond this thickness, the innermost cells are too far from the supply of oxygen and fresh growth medium to thrive. Therefore, it is likely that some cells in the interior of these cell bundles were hypoxia.

The obtained MSC bundles preserved the endogenous ECM which were constituted of proteins, such as collagen type I and type III, fibronectin, laminin and E-CAM. After injection through a needle, we found that MSC bundles retained their activity upon transferring to another growth surface. Cell growth can be regulated by a number of ECM molecules including collagen and fibronectin. These matrix macromolecules are extremely useful for both improving cell adhesion and viability, and controlling the host response that can then mediate cell attachment and spreading.

At retrieval, only a few BrdU-labeled cells were found in the peri-infarcted area in the dissociated-MSC group, whereas a large number of BrdU-labeled cells were identified in the MSC-bundle group. This may be attributed to the fact that MSC bundles had a larger physical size than dissociated MSCs and therefore had a better opportunity to entrap into the interstices of myocardial tissues. Once entrapped into the myocardium, the inherent ECM with MSC bundles could further provide a superior environment for the incorporation of the transplanted cells to the host tissue.

Some MSCs were differentiated into capillaries and arterioles. It was reported that locally delivered MSCs were able to incorporate into newly formed vessels and displayed endothelial or SMC phenotype. Also, MSCs have been shown to express angiogenic growth factors in a paracrine fashion to stimulate neovascularization at the sites of the cell graft. These facts might explain why there was a significantly greater vascular density observed in the MSC-bundle group than in the dissociated-MSC group; consequently contributing toward improved wall thickness and a reduction in the infarct size. The results obtained in our echocardiography and catheterization measurements demonstrated that the MSC-bundle group had a superior heart function to the dissociated-MSC group. Angiogenesis has been shown to contribute to the improvement on myocardial function by the maintenance of the viability of the grafted cells and residual cardiomyocytes.

One aspect of the invention provides a method of treating a target lesion comprising administering stem cells or regenerative cells in at least one living cell bundle configuration, wherein the cell bundle further comprises endogenous extracellular matrices (ECM) for administering into the lesion. In one preferred embodiment, the cell bundle is sized to entrap into interstices of the lesion adapted for offering a favorable ECM environment to retain the administered cells, wherein the cell bundle is in a size range of about 50 mμ to 400 mμ, preferably in a size range of about 100 mμ to 300 mμ.

Using a small animal model, a short-term proof-of-concept study showed the feasibility of this approach. However, a larger animal model in a long-term study would have better simulated the conditions for patients with myocardial infarction. Additionally, the cell population of MSC bundles retained at the injected sites at retrieval was not calculated precisely, as a cell by cell count in such dense conglomerates was impossible.

Forming appropriate segments of living cells sheet or bundle are critical in the medical applications. The cell cut edge should have little effect of any applied cutting energy onto the adjacent living cells. Excess energy may cause cell necrosis and non-contiguity of the cells in the cut cell sheet or bundle. A process for forming segments of living cells sheet is provided for their intended medical use. Some aspects of the invention provide a composite medical device or cells sheet/cluster/bundle that is broken up (that is, segmented) to pieces sized and configured for loading in the delivery instrument. In one embodiment, a process for forming segments of living cells sheet/cluster/bundle is provided by a non-contact means, such as a laser cutting means, ultrasound cutting means, focused ultrasonic cutting means, water jet cutting means, or other energy-assisted cutting means. The non-contact literally means lack of physical contact, since there is no cutting edge which can become contaminated by the material or contaminate the material.

We have disclosed spherical mesenchymal-stem-cell (MSC) bundles inherent with endogenous extracellular matrices (ECM) for direct intramyocardial injection. We also demonstrate that MSC bundles could provide an adequate physical size to entrap into the interstices of muscular tissues and offer a favorable ECM environment to retain the transplanted cells when injected into the peri-infarcted area following experimentally induced myocardial infarction; thus improving the cardiac functions. Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

What is claimed is:

1. A method of treating a target lesion in an animal, the method comprising administering stem cells or regenerative cells to said lesion, said cells being configured in at least one living cell bundle in vitro prior to the administering step.

2. The method of claim 1, wherein said regenerative cells comprise cardiomyocytes.

3. The method of claim 1, wherein said stem cells comprise mesenchymal stem cells or adult multipotent cells.

4. The method of claim 1, wherein said cell bundle further comprises endogenous extracellular matrices (ECM) for administering into said lesion.

5. The method of claim 1, wherein said cell bundle is sized to entrap into interstices of said lesion adapted for offering a favorable ECM environment to retain the administered cells.

6. The method of claim 1, wherein said cell bundle is in a size range of about 50 mμ to 400 mμ.

7. The method of claim 1, wherein said cell bundle is in a size range of about 100 ml to 300 mμ.

8. The method of claim 1, wherein said cell bundle further comprises said cells in a contiguous manner.

9. The method of claim 1, wherein said cell bundle further comprises said cells in a confluent appearance.

10. The method of claim 1, wherein said cell bundle comprises said cells in a spheroid configuration.

11. The method of claim 1, wherein said cell bundle comprises said cells in a cell sheet configuration.

12. The method of claim 1, wherein said lesion comprises an infarcted myocardium.

13. The method of claim 1, wherein said lesion is at a joint.

14. The method of claim 1, wherein said cell bundle comprises a support biomatrix.

15. The method of claim 14, wherein said support biomatrix comprises hydrogel.

16. The method of claim 14, wherein said support biomatrix is biodegradable.

17. The method of claim 14, wherein said lesion is in a breast.

18. The method of claim 1, wherein said at least one cell bundle is sized and configured for loading in a delivery instrument for administering to said target lesion.

19. The method of claim 18, wherein the delivery instrument is a catheter with a needle.

20. The method of claim 18, wherein the delivery instrument is a syringe with a needle.
